(19) United States
(12) Patent Application Publication
(10) Pub. No.: US 2009/0203603 A1
(43) Pub. Date: Aug. 13, 2009

(54) Compositions for the Treatment and Prevention of Nephropathy

(75) Inventors: Alain Baron, San Diego, CA (US); David R. Hathaway, Lincoln, NE (US); Mahesh Mistry, Pepperell, MA (US); Richard J. Roman, Brookfield, WI (US)

Correspondence Address: Intellectual Property Department;Amylin Pharmaceuticals, Inc., 9360 Towne Centre Drive, San Diego, CA, 92121, US

(73) Assignee: AMYLIN PHARMACEUTICALS, INC., San Diego, CA (US)

(21) Appl. No.: 12/338,887

(22) Filed: Dec. 18, 2008

Related U.S. Application Data
(62) Division of application No. 10/741,534, filed on Dec. 19, 2003, now abandoned.
Application No. 10/741,534 is a continuation-in-part of application No. 10/740,146, filed on Dec. 17, 2003.
(60) Provisional application No. 60/434,508, filed on Dec. 17, 2002.
(60) Provisional application No. 60/434,888, filed on Dec. 19, 2002.

(51) Int. Cl. A61K 38/00

(52) U.S. Cl. 514/12

(57) ABSTRACT

Compositions and methods for the prevention and treatment of nephropathy, including hypertensive and diabetic nephropathy, and nephropathy associated with insulin resistance and metabolic syndrome are described. Compositions of the invention include a compound that binds to a receptor for the glucagon like peptide-1, an incretin, a glucagon-like peptide-1 (GLP-1), an exendin, or an analog (including an agonist analog), derivative, or variant of any of them.

### CROSS REFERENCE TO RELATED APPLICATIONS

This application is a divisional of U.S. Ser. No. 10/741,534, filed Dec. 19, 2003, which claims the benefit of priority to U.S. Ser. No. 10/740,146, filed Dec. 17, 2003, which claims priority to U.S. Ser. No. 60/434,508 filed Dec. 17, 2002 and U.S. Ser. No. 60/434,888, filed Dec. 19, 2002, each of which is incorporated herein by reference in their entireties, including all tables, figures, and claims.

### FIELD OF THE INVENTION

This invention related to a composition and method of treating nephropathy, and especially hypertensive and diabetic nephropathy, using GLP-1 and related compounds.

### BACKGROUND OF THE INVENTION

End stage renal disease (ESRD) is a major health problem in the United States. The incidence rate has steadily increased over the past decade, from 155 per million population in 1988 to 296 in 1997. The disease is especially prevalent in racial and ethnic minorities, specifically African Americans, American Indians, Alaskan natives, Native Hawaiians and other Pacific Islanders, and Hispanic Americans.

The four major causes of ESRD include diabetes mellitis (primarily type-2), hypertension, glomerulonephritis, and cystic renal disease. There is significant variabilitiy in the cause of ESRD among the various ethnic and racial groups. For instance, whereas diabetic nephropathy is the predominant cause of ESRD in American Indians/Alaskan Natives, Asian Americans, Native Hawaiians and other Pacific Islanders, Hispanic Americans, and Caucasians, hypertensive nephropathy is the most frequently reported cause of ESRD in African Americans.

Currently, patients with ESRD must either go on dialysis or receive a new kidney through transplant. Every year, high blood pressure causes more than 15,000 new cass of ESRD in the United States.

Diabetic neuropathy is kidney disease that develops as a result of diabetes mellitus. Diabetes affects approximately 5% of the U.S. population. Approximately 25-40% of patients with diabetes ultimately develop diabetic nephropathy, which progresses through five predictable stages, the final stage of which is ESRD, whereby renal replacement therapy (i.e., hemodialysis, peritoneal dialysis, kidney transplantation) is necessary.

Hypertension and diabetes often coexist in the same patient, acting synergistically. The underlying mechanism for nephropathy is not fully understood, but has been postulated to involve a period of glomerular hyperemia followed by a reactive vasoconstriction, leading to glomerular hypertension and subsequent injury. An early manifestation of nephropathy is protein in the urine (e.g. proteinuria), the concentration of which may relate to the degree of kidney damage. Eventually, glomerulosclerosis occurs, leading to a progressive loss of functioning nephrons. The capacity of the kidneys to filter/secrete waste products and maintain electrolyte and water balance is lost, with a rise in the serum creatinine and Blood Urea Nitrogen (BUN) as well as accumulation of excess fluid. At this stage, patients are generally diagnosed with end state renal disease (ESRD).

Individuals with insulin resistance are also at risk, whether or not they have co-existing hypertension, as are patients having so-called “metabolic syndrome.”

The rate of progression of nephropathy can be forestalled by treatment with angiotensin-converting enzyme inhibitors or with the calcium channel blocking drug, verapamil. However, ESRD is inevitable. ESRD, progressing to renal failure, can be treated by dialysis or kidney transplantation. These are expensive therapies that are currently reimbursed by Medicare (irrespective of patient age) with an annual cost of $10 billion. The prevalence of ESRD is increasing.

Accordingly, it can be seen that there is a real and continuing need for an effective treatment for renal damage and nephropathy, including that occurring in conjunction with hypertension, insulin resistance, and/or diabetes. This invention has as its primary object the fulfillment of this need.

### SUMMARY OF THE INVENTION

The invention describes compositions and methods for the prevention and treatment of nephropathy, including hypertensive and diabetic nephropathy, and nephropathy associated with insulin resistance and metabolic syndrome. The invention achieves these ends by improving or preventing worsening of hypertension, endothelial function, renal function, and glomerulosclerosis, among other things. Compositions of the invention include a compound that binds to a receptor for the glucagon like peptide-1, an incretin, a glucagon-like peptide-1 (GLP-1), an exendin, or an agonist, analog (preferably an agonist analog), derivative, variant, or biologically active fragments of any of them.

In one embodiment, the invention provides a method for preventing or treating nephropathy, including hypertensive and diabetic nephropathy, or that related to insulin resistance, comprising administering a compound of the invention.

The invention further provides methods for improving endothelial function in a patient having reduced vasodilatory capacity, or having glomerulosclerosis or any other reduction in glomerular flow. Such improvement in endothelial function serves both to reduce hypertension and to improve the function of the capillaries of the glomerula. In additional embodiments, the molecules of the invention are useful to prevent progression of nephropathy to ESRD, to prevent, slow the progression of, treat or ameliorate proteinuria and/or glomerulosclerosis.

In preferred embodiments of the invention, the compound is a GLP-1 or exendin-3 or exendin-4, or a biologically active analog, derivative, variant, or fragment of them. Preferred dosages are from about 0.001 μg/kg/dose to about 1.0 μg/kg/dose, or at a dose sufficient to achieve a therapeutic plasma level of at least 40 pg/ml.

The means and manner of accomplishing each of the above objectives will become apparent from the detailed description of the invention which follows hereinafter.

### BRIEF DESCRIPTION OF THE DRAWINGS

FIG. 1 presents the effect of rGLP-1 on mean arterial pressure (MAP) in Dahl S rats.

FIG. 2A presents the effect of rGLP-1 on proteinuria concentration in Dahl S rats.

FIG. 2B presents the effect of rGLP-1 on microalbuminuria concentration in Dahl S rats.

FIG. 2C presents the effect of rGLP-1 on plasma creatinine concentration in Dahl S rats.

FIG. 3A presents the effect of rGLP-1 on kidney weight in Dahl S rats.

FIG. 3B presents the effect of rGLP-1 on glomerular injury in Dahl S rats.

FIG. 3C presents the effect of rGLP-1 on the formation of protein casts in outer medulla in Dahl S rats.

FIG. 4 shows the effect of GLP-1 on endothelial function in aortic rings.

### DETAILED DESCRIPTION OF THE INVENTION

The present invention relates to compositions and their uses for the treatment of hypertensive, diabetic, and other types of nephropathy, such as analgesic nephropathy, IgA-nephropathy, ischemic nephropathy, HIV-associated nephropathy, membranous nephropathy, glomerulosclerosis, etc. The invention is especially effective for use in preventing or treating hypertensive and/or diabetic nephropathies, and those occurring or likely to occur in insulin resistant patients with or without co-existing hypertension.

Without wishing to be bound by theory, it is thought that the molecules of the invention act in part by improving insulin resistance, cation balance, hypertension, and/or by facilitating glucose oxidation by cells (including endothelial cells) in the kidney (and elsewhere) rather than oxidation of free fatty acids, leading to an enhanced production of ATP for use by the cell, and reduced oxidative stress on the affected tissue.

Molecules of the invention include compounds that binds to a receptor for the glucagon like peptide-1, incretins, glucagon-like peptide-1s (GLP-1), exendins, or agonists, analogs (preferably an agonist analogs), derivatives, variants, or biologically active fragments of any of them.

As used herein, an “analog” includes any peptide whose sequence was derived from that of the base molecule (e.g., receptor-binding compound, incretin, GLP-1, or exendin), whether or not including insertions, substitutions, extensions, or deletions, preferably having at least 50 or 55% amino acid sequence identity with the base molecule, more preferably having at least 70%, 80%, 90%, or 95% amino acid sequence identity with the base molecule. Such analogs may comprise conservative or non-conservative amino acid substitutions (including non-natural amino acids and L and D forms). An “agonist analog,” is an analog that exhibits at least one characteristic or action of the base molecule, preferably having a potency better than the base molecule, or within five orders of magnitude (plus or minus) of potency compared to the base molecule, more preferably 4, 3, 2, or 1 order of magnitude, when evaluated by art-known measures such as receptor binding/competition studies.

A “derivative” includes any base molecule or analog having a chemical modification within, attached, linked to, or associated with the molecule. Such chemical modifications can include internal linkers (e.g., spacing or structure-inducing) or appended molecules, such as molecular weight-enhancing molecules (e.g., polyethylene glycol (PEG), polyamino acid moieties, etc.), or tissue targeting molecules. Examples of such molecules are known in the art, for example, insulinotropic peptides, including GLP-1 and exendin, modified with a maleimide group are described in U.S. Pat. No. 6,593,295, incorporated herein by reference.

A “variant” includes any modification to the base molecule, analog or variant not encompassed in the terms “analog” and “derivative,” as would be known to a person of ordinary skill in the art. For example, variants may include proforms or chimeras of a selected molecule. Small molecules are included in the compounds useful in the invention to the extent that they bind to a receptor for GLP-1 or exendin, or have nephropathy-preventing or -treating characteristics as described herein. It is understood that not all of the peptide molecules described as incretins, glucagon-like peptide-1 (GLP-1), exendins, or analogs, derivatives, or variants may bind to a receptor for GLP-1, although they are still useful in the invention by virtue of a pharmacology not dependent on a known GLP-1 receptor. These molecules may still possess the desired biological activities described herein, for example GLP-1(9-36), and agonists, analogs, derivatives, and variants thereof. Other exemplary compounds encompassed within the scope of the invention include those described in U.S. Pat. Nos. 6,569,832; 6,528,486; 6,514,500; 6,458,924; 6,451,987; 6,451,974; 6,268,343, all herein incorporated by reference.

An example of a base molecule of the invention, as the term is used above, is GLP-1, also known as glucagon-like peptide-1 [7-36], whether or not amided (often GLP-1 [7-36]NH₂), a product of the proglucagon gene having the amino acid sequence His Ala Glu Gly Thr Phe Thr Ser Asp Val Ser Ser Tyr Leu Glu Gly Gln Ala Ala Lys Glu Phe Ile Ala Trp Leu Val Lys Gly Arg-[optional NH₂] (SEQ ID NO: 1). GLP-1 is a hormone produced by L-type cells in the intestine, and is released following ingestion of a meal. GLP-1 improves insulin resistance and glucose utilization in patients with type-2 diabetes by increasing the secretion of insulin and by inhibiting the secretion of glucagon. Receptors for GLP-1 are expressed in pancreatic islet cells, the gastrointestinal tract, and in the lung, heart, central nervous system and kidney. GLP-1 reportedly produces a variety of biological effects (e.g., Orskov, et al., Diabetes, 42:658-61, 1993; D'Alessio, et al., J. Clin. Invest., 97:133-38, 1996, Williams B, et al., J Clin Endocrinol Metab 81 (1): 327-32, 1996; Wettergren A, et al., Dig Dis Sci 38 (4): 665-73, 1993; Schjoldager B T, et al., Dig Dis Sci 34 (5): 703-8, 1989; O'Halloran D J, et al., J Endocrinol 126 (1): 169-73, 1990; Wettergren A, et al., Dig Dis Sci 38 (4): 665-73, 1993). GLP-1[7-37], which has an additional glycine residue at its carboxy terminus, also stimulates insulin secretion in humans (Orskov, et al., Diabetes, 42:658-61, 1993).

Compositions of the invention include GLP-1 agonist analogs. By “agonist analog” is meant a compound that mimics at least one effect of GLP-1. This definition of agonist analog could include compounds that bind to a receptor or receptors where GLP-1 causes the particular effect. Certain GLP-1 analogs with agonist activity are described in Chen et al., U.S. Pat. No. 5,512,549, issued Apr. 30, 1996, entitled Glucagon-Like Insulinotropic Peptide Analogs, Compositions and Methods of Use. Other GLP-1 analogs with agonist activity are described in Johnson et al., U.S. Pat. No. 5,574,008, issued Nov. 12, 1996, entitled, Biologically Active Fragments of Glucagon-Like Insulinotropic Peptide. Still other GLP-1 analogs with agonist activity are described in Buckley et al., U.S. Pat. No. 5,545,618, issued Aug. 13, 1996, entitled GLP-1 Analogs Useful for Diabetes Treatment. All three referenced U.S. patents are incorporated herein by this reference. The present invention includes the use of recombinant human GLP-1 analogs and GLP-1 analogs derived from other species, whether recombinant or otherwise synthetic.

In certain aspects, the GLP-1 agonist analogs used in the methods of the present invention can be GLP-1(7-34) and GLP-1(7-35), as disclosed in U.S. Pat. No. 5,118,666, herein incorporated by reference, as well as GLP-1(7-37) as disclosed in U.S. Pat. No. 5,120,712, herein incorporated by reference. Also included are GLP-1 analogs having a reduced tendency to aggregate such as those described in WO 01/98331; GLP-1 analogs that have N-terminal truncation, U.S. Pat. No. 5,574,008; GLP-1 analogs with attached acyl groups, U.S. Pat. No. 5,512,549; and GLP-1 analogs that are amidated, WO 02/48192; and GLP-1 analogs of U.S. patent application Ser. No. 10/276,772, all of which are incorporated by reference.

Additional exemplary analogs include GLP-1 analogs modified at position 8, e.g., U.S. Pat. No. 5,981,488, incorporated by reference. In brief, analogs include those of formula (XI), R₁-X-Glu-Gly-Thr-Phe-Thr-Ser-Asp-Val-Ser-Ser-Tyr-Leu-Y-Gly-Gln-Ala-Ala-Lys-Z-Phe-Ile-Ala-Trp-Leu-Val-Lys-Gly-Arg-R₂ (SEQ ID NO:33) or a pharmaceutically acceptable salt thereof, wherein:
R₁ is selected from the group consisting of His, D-histidine, desamino-histidine, 2-amino-histidine, beta.-hydroxy-histidine, homohistidine, alpha-fluoromethyl-histidine, and alpha-methyl-histidine;
X is selected from the group consisting of Met, Asp, Lys, Thr, Leu, Asn, Gln, Phe, Val, and Tyr
Y and Z are independently selected from the group consisting of Glu, Gln, Ala, Thr, Ser, and Gly, and;
R₂ is selected from the group consisting of NH₂, and Gly-OH; provided that, if R₁ is His, X is Val, Y is Glu, and Z is Glu, then R₂ is NH₂.

V8-GLP-1 and other position 8 analogs can be found in U.S. Pat. No. 5,705,483, incorporated by reference. In brief, analogs include those of formula (XII), R₁-X-Glu-Gly-Thr-Phe-Thr-Ser-Asp-Val-Ser-Ser-Tyr-Leu-Y-Gly-Gln-Ala-Ala-Lys-Z-Phe-Ile-Ala-Trp-Leu-Val-Lys-Gly-Arg-R₂ (SEQ ID NO: 34) wherein: R₁ is selected from the group consisting of L-histidine, D-histidine, desamino-histidine, 2-amino-histidine, beta-hydroxy-histidine, homohistidine, alpha-fluoromethyl-histidine, and alpha-methyl-histidine;
X is selected from the group consisting of Ala, Gly, Val, Thr, Ile, and alpha-methyl-Ala;
Y is selected from the group consisting of Glu, Gln, Ala, Thr, Ser, and Gly;
Z is selected from the group consisting of Glu, Gln, Ala, Thr, Ser, and Gly;
R₂ is selected from the group consisting of NH₂, and Gly-OH; providing that the compound has an isoelectric point in the range from about 6.0 to about 9.0 and further providing that when R₁ is His, X is Ala, Y is Glu, and Z is Glu, R₂ must be NH₂.

In other aspects, the GLP-1 agonist analogs are variants or analogs of GLP-1 known in the art, such as, for example, Gln⁹-GLP-1(7-37), D-Gln⁹-GLP-1(7-37), acetyl-Lys⁹-GLP-1(7-37), Thr¹⁶-Lys¹⁸-GLP-1(7-37), and Lys¹⁸-GLP-1(7-37). Derivatives of GLP-1 are also contemplated in the present invention and include, for example, acid addition salts, carboxylate salts, lower alkyl esters, and amides (see, e.g., WO91/11457). Generally, but not necessarily for use in this invention, the various forms of GLP-1 are known to stimulate insulin secretion (insulinotropic action) and cAMP formation (see, e.g., Mojsov, S., Int. J. Peptide Protein Research, 40:333-343 (1992)).

In still other aspects, the present invention contemplates GLP-1 agonists of the general formula (I):

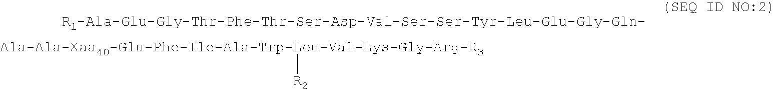

wherein R₁ is selected from the group consisting of 4-imidazopropionyl (des-amino-histidyl), 4-imidazoacetyl, or 4-imidazo-alpha,alpha dimethyl-acetyl;
R₂ is selected from the group consisting of C₆-C₁₀ unbranched acyl, or is absent;
R₃ is selected from the group consisting of Gly-OH or NH₂; and,
Xaa₄₀ is Lys or Arg.

In one embodiment, the GLP-1 agonists are naturally-occurring GLP-1(7-37) that arise from adding various R groups via a peptide bond to the amino terminus of the peptide portion of Formula I (SEQ ID NO:2). Optionally, further compounds of the invention are made by acylating the epsilon amino group of the Lys34 residue and by making limited amino acid substitutions at position 26 or by altering the carboxy terminus.

It should be noted that for the above formula, the nomenclature scheme used is that which has been developed around processed forms of GLP-1. In this scheme, the amino terminus of the known GLP-1(7-37) OH has been assigned number 7 and the carboxy terminus number 37. Therefore, the first Ala residue of Formula I corresponds to residue 8 of GLP-1(7-37)OH. Likewise Xaa₄₀ in Formula I corresponds to residue 26 of GLP-1(7-37)OH, and so forth.

In still other aspects, the present invention provides biologically-active GLP-1 fragments of formula (II):

(SEQ ID NO:3)

R₄-Ser-Tyr-Leu-Glu-Gly-Gln-Ala-Ala-Lys-Glu-Phe-
Ile-Ala-Trp-Leu-Val-Xaa₄₁-Gly-Arg-R₅

wherein R₄ is selected from the group consisting of:

a) H₂ N;
b) H₂ N-Ser;
c) H₂ N-Val-Ser;
d) H₂ N-Asp-Val-Ser;

(SEQ ID NO:4)

e) H₂ N-Ser-Asp-Val-Ser;

(SEQ ID NO:5)

f) H₂ N-Thr-Ser-Asp-Val-Ser;

(SEQ ID NO:6)

g) H₂ N-Phe-Tbr-Ser-Asp-Val-Ser;

(SEQ ID NO:7)

h) H₂ N-Thr-Phe-Thr-Ser-Asp-Val-Ser;

(SEQ ID NO:8)

i) H₂ N-Gly-Thr-Phe-Thr-Ser-Asp-Val-Ser;

(SEQ ID NO:9)

j) H₂ N-Glu-Gly-Thr-Phe-Thr-Ser-Asp-Val-Ser;
or

(SEQ ID NO:10)

k) H₂ N-Ala-Glu-Gly-Thr-Phe-Thr-Ser-Asp-Val-Ser;

Xaa₄₁ is selected from the group consisting of Lys or Arg; and

wherein R₅ is selected from the group consisting of NH₂, OH, Gly-NH₂, or Gly-OH.

In still other aspects, the invention provides modified forms of the GLP-1(7-34); (7-35); (7-36) or (7-37) human peptide or the C-terminal amidated forms thereof. The native peptides have the amino acid sequence (SEQ ID NO: 11):

7 10 15 20 25
H-A-E-G-T-F-T-S-D-V-S-S-Y-L-E-G-Q-A-A-K-E-F-
30 37
I-A-W-L-V-K-(G)-(R)-(G)

wherein (G), (R), and (G) are present or absent depending on the indicated chain length. The modified forms contain one or more alterations of the native structure and are of improved ability for therapeutic use. Either the modified forms have greater potency than glucagon to potentiate insulin secretion or enhanced stability in plasma or both.

The analogs of the invention may have the foregoing sequence, or a C-terminal amide thereof, with at least one modification of SEQ ID NO: 11, selected from the group consisting of:

(a) substitution of a neutral amino acid, arginine, or a D form of lysine for lysine at position 26 and/or 34 and/or a neutral amino acid, lysine, or a D form of arginine for arginine at position 36;
(b) substitution of an oxidation-resistant amino acid for tryptophan at position 31;
(c) substitution according to at least one of:

Y for V at position 16;

K for S at position 18;

D for E at position 21;

S for G at position 22;

R for Q at position 23;

R for A at position 24; and

Q for K at position 26;

(d) a substitution comprising at least one of:

an alternative small neutral amino acid for A at position 8;

an alternative acidic amino acid or neutral amino acid for E at position 9;

an alternative neutral amino acid for G at position 10; and

an alternative acidic amino acid for D at position 15; and

(e) substitution of an alternative neutral amino acid or the D or N-acylated or alkylated form of histidine for histidine at position 7.

With respect to modifications (a), (b), (d) and (e), the substituted amino acids may be in the D form, as indicated by a superscript †, e.g., C^{†}. The amino acids substituted at position 7 can also be in the N-acylated or N-alkylated forms.

In another aspect, the invention is directed to peptides which show enhanced degradation resistance in plasma as compared to GLP-1(7-37) wherein this enhanced resistance to degradation is defined as set forth below. In these analogs, any of the above-mentioned truncated forms of GLP-1(7-34) to GLP-1(7-37) or their C-terminal amidated form is modified by

(a) substitution of a D-neutral or D-acidic amino acid for H at position 7, or
(b) substitution of a D-amino acid for A at position 8, or
(c) both, or
(d) substitution of an N-acylated or N-alkylated form of any naturally occurring amino acid for H at position 7.

Thus, analogs of the invention which are resistant to degradation include (N-acyl (1-6C) AA)⁷ GLP-1(7-37) and (N-alkyl (1-6C) AA)⁷ GLP-1(7-37) wherein when AA is a lysyl residue, one or both nitrogens may be alkylated or acylated. AA symbolizes any amino acid consistent with retention of insulin stimulating activity.

For substitutions of D-amino acids in the 7 and 8 positions of SEQ ID NO: 11, the D residue of any acidic or neutral amino acid can be used at position 7 and of any amino acid at position 8, again consistent with insulin stimulating activity. Either or both of position 7 and 8 can be substituted by a D-amino acid; the D-amino acid at position 7 can also be acylated or alkylated as set forth above. These modified forms are applicable not only to GLP-1(7-37) but also the shorter truncated analogs as set forth above.

Other modified GLP-1s, as well as exendins, useful in the practice of the claimed invention can be found in U.S. Pat. No. 6,528,486, which is incorporated by reference. Further, agonists of glucagon-like peptide that exhibit activity through a GLP-1(7-36)amide receptor have been described. See EP 0708179 A2; Hjorth et al., J. Biol. Chem. 269; 30121 (1994); Siegel et al., Amer. Diabetes Assoc. 57^{th} Scientific Session, Boston (1997); Hareter et al., Amer. Diabetes Assoc. 57^{th} Scientific Session, Boston (1997); Adelhorst et al., J. Biol. Chem. 269, 6275 (1994); Deacon et al., 16^{th} International Diabetes Federation Congress Abstracts, Diabetologia Supplement (1997); Irwin et al., Proc. Natl. Acad. Sci. USA 94; 7915 (1997); Mojsov, Int. J. Peptide Protein Res. 40; 333 (1992). Goke & Byrne, Diabetic Medicine 13; 854 (1996). Recent publications disclose Black Widow GLP-1 and Ser² GLP-1. See Holz & Hakner, Comp. Biochem. Physiol., Part B 121; 177 (1998) and Ritzel et al., J. Endocrinol 159; 93 (1998).

As previously stated, GLP-1 analogs, as well as exendin analogs, may be peptides containing one or more amino acid substitutions, additions, extensions, or deletions, compared with GLP-1(7-36), exendin-4 or exendin-3. In one embodiment, the number of substitutions, deletions, or additions is 30 amino acids or less, 25 amino acids or less, 20 amino acids or less, 15 amino acids or less, 10 amino acids or less, 5 amino acids or less or any integer in between these amounts. In one aspect of the invention, the substitutions include one or more conservative substitutions. A “conservative” substitution denotes the replacement of an amino acid residue by another, biologically active similar residue as is well known in the art. Examples of conservative substitutions include the substitution of one hydrophobic residue, such as isoleucine, valine, leucine, or methionine for another, or the substitution of one polar residue for another, such as the substitution of arginine for lysine, glutamic for aspartic acids, or glutamine for asparagine, and the like.

It is further understood that GLP-1 analogs include the above described peptides which have been chemically derivatized or altered, for example, peptides with non-natural amino acid residues (e.g., taurine, β- and γ-amino acid residues and D-amino acid residues), C-terminal functional group modifications, such as amides, esters, and C-terminal ketone modifications and N-terminal functional group modifications, such as acylated amines, Schiff bases, or cyclization, as found, for example, in the amino acid pyroglutamic acid. Exendin analogs, including those described below, may have similar modifications.

Other compositions of the invention include exendins, which refer to naturally occurring exendin peptides that are found in Gila-monster and related peptides. Preferred exendins include exendin-3 (SEQ ID NO: 12), which is present in the salivary secretions of Heloderma horridum, exendin-4 (SEQ ID NO: 14), which is a peptide present in the salivary secretions of Heloderma suspectum (Eng, J., et al., J. Biol. Chem., 265:20259-62, 1990; Eng., J., et al., J. Biol. Chem., 267:7402-05, 1992), and agonists, analogs, derivatives, or variants of either of them, as well as biologically active fragments thereof. Exendin-4, as it occurs in the salivary secretions of the Gila monster, is an amidated peptide. However, it should be understood that the terms “exendin,” “exendin-3,” and “exendin-4” refer to both the amidated form of the peptide and the acid form of the peptide. Likewise, reference to GLP-1 generally refers to the amidated 7-36 molecule, but it is also intended to include non-amidated molecules, and analogs, derivatives and variants of these peptides may likewise be amidated or not.

“Exendin agonist” refers to compounds that mimic any effect of an exendin by binding to a receptor or receptors where a naturally occurring exendin exerts an effect. Exendin “agonist activity” in this context means having a biological activity of an exendin, such as those described herein; but it is understood that the activity of the agonist can be either less potent or more potent than the native exendin.

Exendin-4 is a 39-amino acid polypeptide. Certain sequences of molecules of the invention are compared in Table 1.

TABLE 1

```
a. H A E G T F T S D V S S Y L E G Q A A K E F I A
      W L V K G R (NH₂)
b. H S D G T F T S D L S K Q M E E E A V R L F I E
      W L K N G G P S S G A P P P S (NH₂)
c. D L S K Q M E E E A V R L F I E W L K N G G P S
      S G A P P P S (NH₂)
d. H G E G T F T S D L S K Q M E E E A V R L F I E
      W L K N G G P S S G A P P P S (NH₂)
e. H S D A T F T A E Y S K L L A K L A L Q K Y L E
      S I L G S S T S P R P P S S
f. H S D A T F T A E Y S K L L A K L A L Q K Y L E
      S I L G S S T S P R P P S
g. H S D A I F T E E Y S K L L A K L A L Q K Y L A
      S I L G S R T S P P P (NH₂)
h. H S D A I F T Q Q Y S K L L A K L A L Q K Y L A
      S I L G S R T S P P P (NH₂)
```

a = GLP-1(7-36) (NH₂) [SEQ ID NO: 1].
b = exendin 3 (NH₂) [SEQ ID NO: 12].
c = exendin 4 (9-39)(NH₂) [SEQ ID NO: 13] (an antagonist of exendin-4 and GLP-1)
d = exendin 4 (NH₂) [SEQ ID NO: 14].
e = helospectin I [SEQ ID NO: 15].
f = helospectin II [SEQ ID NO: 16].
g = helodermin (NH₂) [SEQ ID NO: 17].
h = Q⁸, Q⁹ helodermin (NH₂) [SEQ ID NO: 18].

Various experiments have compared the biologic actions of exendin-4 and GLP-1 and demonstrated a more favorable spectrum of properties for exendin-4 for certain indications. Exendin has been shown to lower plasma glucose, lower HbA_{1c} (a measure of glycosylated hemoglobin used to evaluate plasma glucose levels), improve insulin sensitivity, and improve insulin response to glucose. Higher plasma glucose concentrations are associated with greater glucose-lowering effects, thus the observed glucose lowering effect of exendin-4 appears to be glucose-dependent, and minimal if animals are already euglycemic. Degradation studies with exendin-4 compared to GLP-1 indicate that exendin-4 is relatively resistant to degradation.

As used in this specification, the term “exendin agonist” includes any molecules, whether they be peptides, peptide mimetics, or other chemical compounds, that bind to or activate a receptor or receptors at which exendin exerts an effect, including one of those described above. Exendin agonists may include molecules having insulinotropic activity and that may bind a GLP-1 receptor molecule in in vitro assays and induce second messenger activity on, inter alia, insulin producing β-cells, but these actions are not necessary for an exendin agonist or analog to be useful in the instant invention.

The structure activity relationship (SAR) of exendin was investigated for structures that may relate to the activity of exendin, for its stability to metabolism, and for improvement of its physical characteristics, especially as it pertains to peptide stability and to amenability to alternative delivery systems, and various exendin agonist peptide compounds have been invented. Exendin agonists include exendin analogs with agonist activity in which one or more naturally or non-naturally occurring amino acids are added, inserted, eliminated or replaced with another amino acid(s). Preferred exendin analogs are peptide analogs of exendin-4.

Exendin analogs include peptides that are encoded by polynucleotides that express biologically active exendin analogs with agonist activity, and which are functional in the invention, as defined herein. For instance, exendin analogs usefule in the invention may be peptides containing one or more amino acid substitutions, extensions, additions or deletions, compared with exendin-4 or exendin-3. In one embodiment, the number of substitutions, extensions, deletions, or additions is 30 amino acids or less, 25 amino acids or less, 20 amino acids or less, 15 amino acids or less, 10 amino acids or less, 5 amino acids or less or any integer in between these amounts. In one aspect of the invention, the substitutions include one or more conservative substitutions. Exendin analogs, which include chemically derivatized or altered compounds and peptides having a preferred amino acid homology to SEQ ID NOs: 12 and 14 have been previously described and are contemplated to be within the scope of the claimed invention.

Novel exendin analogs with agonist activity are described in PCT Application Serial No. PCT/US98/16387 filed Aug. 6, 1998, entitled “Novel Exendin Agonist Compounds,” which claims the benefit of U.S. Patent Application Ser. No. 60/055,404, filed Aug. 8, 1997, both of which are herein incorporated by reference.

Other novel exendin analogs with agonist activity are described in PCT Application Serial No. PCT/US98/24210, filed Nov. 13, 1998, entitled “Novel Exendin Agonist Compounds,” which claims the benefit of U.S. Provisional Application No. 60/065,442 filed Nov. 14, 1997, both of which are herein incorporated by reference.

Still other novel exendin analogs with agonist activity are described in PCT Application Serial No. PCT/US98/24273, filed Nov. 13, 1998, entitled “Novel Exendin Agonist Compounds,” which claims the benefit of U.S. Provisional Application No. 60/066,029 filed Nov. 14, 1997, both of which are herein incorporated by reference.

Still other exendin analogs with agonist activity are described in PCT Application Serial No. PCT/US97/14199, filed Aug. 8, 1997, entitled “Methods for Regulating Gastrointestinal Activity,” which is a continuation-in-part of U.S. patent application Ser. No. 08/694,954 filed Aug. 8, 1996, both of which are hereby incorporated by reference.

Still other exendin analogs with agonist activity are described in PCT Application Serial No. PCT/US98/00449, filed Jan. 7, 1998, entitled “Use of Exendins and Agonists Thereof for the Reduction of Food Intake,” which claims priority to U.S. Provisional Application No. 60/034,905 filed Jan. 7, 1997, both of which are hereby incorporated by reference.

Exendin agonist activity can be evaluated, for example, by ascertaining activity in the assays incorporated by reference in the referenced applications. Effects of exendins or exendin agonists can be identified, evaluated, or screened for, using the methods described therein, or other art-known or equivalent methods for determining the effects of exendin. Screening assays for potential exendin agonist compounds or candidate exendin agonist compounds, may include an in vitro GLP-1 receptor competitive assay or direct binding screen, or an activity screen, such as increased cAMP production or insulin synthesis.

Certain preferred exendin analogs with agonist activity include:

Exendin-4 (1-30)
[SEQ ID NO:19: His Gly Glu Gly Thr Phe Thr Ser Asp
Leu Ser Lys Gln Met Glu Glu Glu Ala Val Arg Leu
Phe Ile Glu Trp Leu Lys Asn Gly Gly];
Exendin-4 (1-30) amide
[SEQ ID NO:20: His Gly Glu Gly Thr Phe Thr Ser Asp
Leu Ser Lys Gln Met Glu Glu Glu Ala Val Arg Leu
Phe Ile Glu Trp Leu Lys Asn Gly Gly-NH₂];
Exendin-4 (1-28) amide
[SEQ ID NO:21: His Gly Glu Gly Thr Phe Thr Ser Asp
Leu Ser Lys Gln Met Glu Glu Glu Ala Val Arg Leu
Phe Ile Glu Trp Leu Lys Asn-NH₂];
¹⁴Leu, ²⁵Phe exendin-4 amide
[SEQ ID NO:22: His Gly Glu Gly Thr Phe Thr Ser Asp
Leu Ser Lys Gln Leu Glu Glu Glu Ala Val Arg Leu
Phe Ile Glu Phe Leu Lys Asn Gly Gly Pro Ser Ser
Gly Ala Pro Pro Pro Ser-NH₂];
¹⁴Leu, ²⁵Phe exendin-4 (1-28) amide
[SEQ ID NO:23: His Gly Glu Gly Thr Phe Thr Ser Asp
Leu Ser Lys Gln Leu Glu Glu Glu Ala Val Arg Leu
Phe Ile Glu Phe Leu Lys Asn-NH₂];
and
¹⁴Leu, ²²Ala, ²⁵Phe exendin-4 (1-28) amide
[SEQ ID NO:24: His Gly Glu Gly Thr Phe Thr Ser Asp
Leu Ser Lys Gln Leu Glu Glu Glu Ala Val Arg Leu
Ala Ile Glu Phe Leu Lys Asn-NH₂].

Also included within the scope of the present invention are pharmaceutically acceptable salts of the compounds of formula (III-X) and pharmaceutical compositions including said compounds and salts thereof.

### Formula III

Exendin analogs with agonist activity also include those described in U.S. Ser. No. 09/554,533, including compounds of the formula (III) [SEQ ID NO:25]:

Xaa₁ Xaa₂ Xaa₃ Gly Xaa₅ Xaa₆ Xaa₇ Xaa₈ Xaa₉ Xaa₁₀
Xaa₁₁ Xaa₁₂ Xaa₁₃ Xaa₁₄ Xaa₁₅ Xaa₁₆ Xaa₁₇ Ala
Xaa₁₉ Xaa₂₀ Xaa₂₁ Xaa₂₂ Xaa₂₃ Xaa₂₄ Xaa₂₅ Xaa₂₆
Xaa₂₇ Xaa₂₈-Z₁;

wherein

Xaa₁ is His, Arg or Tyr;

Xaa₂ is Ser, Gly, Ala or Thr;

Xaa₃ is Asp or Glu;

Xaa₅ is Ala or Thr;

Xaa₆ is Ala, Phe, Tyr or naphthylalanine;

Xaa₇ is Thr or Ser;

Xaa₈ is Ala, Ser or Thr;

Xaa₉ is Asp or Glu;

Xaa₁₀ is Ala, Leu, Ile, Val, pentylglycine or Met;

Xaa₁₁ is Ala or Ser;

Xaa₁₂ is Ala or Lys;

Xaa₁₃ is Ala or Gln;

Xaa₁₄ is Ala, Leu, Ile, pentylglycine, Val or Met;

Xaa₁₅ is Ala or Glu;

Xaa₁₆ is Ala or Glu;

Xaa₁₇ is Ala or Glu;

Xaa₁₉ is Ala or Val;

Xaa₂₀ is Ala or Arg;

Xaa₂₁ is Ala or Leu;

Xaa₂₂ is Ala, Phe, Tyr or naphthylalanine;

Xaa₂₃ is Ile, Val, Leu, pentylglycine, tert-butylglycine or Met;

Xaa₂₄ is Ala, Glu or Asp;

Xaa₂₅ is Ala, Trp, Phe, Tyr or naphthylalanine;

Xaa₂₆ is Ala or Leu;

Xaa₂₇ is Ala or Lys;

Xaa₂₈ is Ala or Asn;

Z₁ is —OH,
—NH₂
Gly-Z₂,
Gly Gly-Z₂,
Gly Gly Xaa₃₁-Z₂,
Gly Gly Xaa₃₁ Ser-Z₂,
Gly Gly Xaa₃₁ Ser Ser-Z₂,
Gly Gly Xaa₃₁ Ser Ser Gly-Z₂,
Gly Gly Xaa₃₁ Ser Ser Gly Ala-Z₂,
Gly Gly Xaa₃₁ Ser Ser Gly Ala Xaa₃₆-Z₂,
Gly Gly Xaa₃₁ Ser Ser Gly Ala Xaa₃₆ Xaa₃₇-Z₂ or
Gly Gly Xaa₃₁ Ser Ser Gly Ala Xaa₃₆ Xaa₃₇ Xaa₃₈-Z₂;
Xaa₃₁, Xaa₃₆, Xaa₃₇ and Xaa₃₈ are independently Pro, homoproline, 3Hyp, 4Hyp, thioproline, N-alkylglycine, N-alkylpentylglycine or N-alkylalanine; and
Z₂ is —OH or —NH₂;

provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala.

Preferred N-alkyl groups for N-alkylglycine, N-alkylpentylglycine and N-alkylalanine include lower alkyl groups preferably of 1 to about 6 carbon atoms, more preferably of 1 to 4 carbon atoms.

Preferred exendin analogs include those wherein Xaa₁ is His or Tyr. More preferably Xaa₁ is His.

Preferred are those compounds wherein Xaa₂ is Gly.

Preferred are those compounds wherein Xaa₁₄ is Leu, pentylglycine or Met.

Preferred compounds are those wherein Xaa₂₅ is Trp or Phe.

Preferred compounds are those where Xaa₆ is Phe or naphthylalanine; Xaa₂₂ is Phe or naphthylalanine and Xaa₂₃ is Ile or Val.

Preferred are compounds wherein Xaa₃₁, Xaa₃₆, Xaa₃₇ and Xaa₃₈ are independently selected from Pro, homoproline, thioproline and N-alkylalanine.

Preferably Z₁ is —NH₂.

Preferably Z₂ is —NH₂.

According to one aspect, preferred are compounds of formula (III) wherein Xaa₁ is His or Tyr, more preferably His; Xaa₂ is Gly; Xaa₆ is Phe or naphthylalanine; Xaa₁₄ is Leu, pentylglycine or Met; Xaa₂₂ is Phe or naphthylalanine; Xaa₂₃ is Ile or Val; Xaa₃₁, Xaa₃₆, Xaa₃₇ and Xaa₃₈ are independently selected from Pro, homoproline, thioproline or N-alkylalanine. More preferably Z₁ is —NH₂.

According to an especially preferred aspect, especially preferred compounds include those of formula (III) wherein: Xaa₁ is His or Arg; Xaa₂ is Gly or Ala; Xaa₃ is Asp or Glu; Xaa₅ is Ala or Thr; Xaa₆ is Ala, Phe or nephthylalaine; Xaa₇ is Thr or Ser; Xaa₈ is Ala, Ser or Thr; Xaa₉ is Asp or Glu; Xaa₁₀ is Ala, Leu or pentylglycine; Xaa₁₁ is Ala or Ser; Xaa₁₂ is Ala or Lys; Xaa₁₃ is Ala or Gln; Xaa₁₄ is Ala, Leu or pentylglycine; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Phe or naphthylalanine; Xaa₂₃ is Ile, Val or tert-butylglycine; Xaa₂₄ is Ala, Glu or Asp; Xaa₂₅ is Ala, Trp or Phe; Xaa₂₆ is Ala or Leu; Xaa₂₇ is Ala or Lys; Xaa₂₈ is Ala or Asn; Z₁ is —OH, —NH₂, Gly-Z₂, Gly Gly-Z₂, Gly Gly Xaa₃₁-Z₂, Gly Gly Xaa₃₁ Ser-Z₂, Gly Gly Xaa₃₁ Ser Ser-Z₂, Gly Gly Xaa₃₁ Ser Ser Gly-Z₂, Gly Gly Xaa₃₁ Ser Ser Gly Ala-Z₂, Gly Gly Xaa₃₁ Ser Ser Gly Ala Xaa₃₆-Z₂, Gly Gly Xaa₃₁ Ser Ser Gly Ala Xaa₃₆ Xaa₃₇-Z₂, Gly Gly Xaa₃₁ Ser Ser Gly Ala Xaa₃₆ Xaa₃₇ Xaa₃₈-Z₂; Xaa₃₁, Xaa₃₆, Xaa₃₇ and Xaa₃₈ being independently Pro homoproline, thioproline or N-methylalanine; and Z₂ being —OH or —NH₂; provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala. Especially preferred compounds include those set forth in PCT application Serial No. PCT/US98/24210, filed Nov. 13, 1998, entitled “Novel Exendin Agonist Compounds” identified therein as compounds 2-23.

According to an especially preferred aspect, provided are compounds where Xaa₁₄ is Leu, Ile, Val or pentylglycine, more preferably Leu or pentylglycine, and Xaa₂₅ is Phe, Tyr or naphthylalanine, more preferably Phe or naphthylalanine. These compounds will be less susceptive to oxidative degration, both in vitro and in vivo, as well as during synthesis of the compound.

### Formula IV

Exendin analogs with agonist activity also include those described in U.S. Provisional application Ser. No. 09/554,531, including compounds of the formula (IV)[SEQ ID NO:26]:

Xaa₁ Xaa₂ Xaa₃ Xaa₄ Xaa₅ Xaa₆ Xaa₇ Xaa₈ Xaa₉ Xaa₁₀
Xaa₁₁ Xaa₁₂ Xaa₁₃ Xaa₁₄ Xaa₁₅ Xaa₁₆ Xaa₁₇ Ala
Xaa₁₉ Xaa₂₀ Xaa₂₁ Xaa₂₂ Xaa₂₃ Xaa₂₄ Xaa₂₅ Xaa₂₆
Xaa₂₇ Xaa₂₈-Z₁;

wherein:

Xaa₁ is His, Arg, Tyr, Ala, Norval, Val or Norleu;

Xaa₂ is Ser, Gly, Ala or Thr;

Xaa₃ is Ala, Asp or Glu;

Xaa₄ is Ala, Norval, Val, Norleu or Gly;

Xaa₅ is Ala or Thr;

Xaa₆ is Phe, Tyr or naphthylalanine;

Xaa₇ is Thr or Ser;

Xaa₈ is Ala, Ser or Thr;

Xaa₉ is Ala, Norval, Val, Norleu, Asp or Glu;

Xaa₁₀ is Ala, Leu, Ile, Val, pentylglycine or Met;

Xaa₁ is Ala or Ser;

Xaa₁₂ is Ala or Lys;

Xaa₁₃ is Ala or Gln;

Xaa₁₄ is Ala, Leu, Ile, pentylglycine, Val or Met;

Xaa₁₅ is Ala or Glu;

Xaa₁₆ is Ala or Glu;

Xaa₁₇ is Ala or Glu;

Xaa₁₉ is Ala or Val;

Xaa₂₀ is Ala or Arg;

Xaa₂₁ is Ala or Leu;

Xaa₂₂ is Phe, Tyr or naphthylalanine;

Xaa₂₃ is le, Val, Leu, pentylglycine, tert-butylglycine or Met;

Xaa₂₄ is Ala, Glu or Asp;

Xaa₂₅ is Ala, Trp, Phe, Tyr or naphthylalanine;

Xaa₂₆ is Ala or Leu;

Xaa₂₇ is Ala or Lys;

Xaa₂₈ is Ala or Asn;

Z₁ is —OH,
—NH₂,
Gly-Z₂,
Gly Gly-Z₂,
Gly Gly Xaa₃′-Z₂,
Gly Gly Xaa₃₁ Ser-Z₂,
Gly Gly Xaa₃₁ Ser Ser-Z₂,
Gly Gly Xaa₃₁ Ser Ser Gly-Z₂,
Gly Gly Xaa₃₁ Ser Ser Gly Ala-Z₂,
Gly Gly Xaa₃₁ Ser Ser Gly Ala Xaa₃₆-Z₂,
Gly Gly Xaa₃₁ Ser Ser Gly Ala Xaa₃₆ Xaa₃₇-Z₂,
Gly Gly Xaa₃₁ Ser Ser Gly Ala Xaa₃₆ Xaa₃₇ Xaa₃₈-Z₂ or
Gly Gly Xaa₃₁ Ser Ser Gly Ala Xaa₃₆ Xaa₃₇ Xaa₃₈ Xaa₃₉-Z₂;
Xaa₃₁, Xaa₃₆, Xaa₃₇ and Xaa₃₈ are independently Pro, homoproline, 3Hyp, 4Hyp, thioproline, N-alkylglycine, N-alkylpentylglycine or N-alkylalanine; and
Z₂ is —OH or —NH₂;

provided that no more than three of Xaa₃, Xaa₄, Xaa₅, Xaa₆, Xaa₈, Xaa₉, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala; and provided also that, if Xaa₁ is His, Arg or Tyr, then at least one of Xaa₃, Xaa₄ and Xaa₉ is Ala.

Preferred N-alkyl groups for N-alkylglycine, N-alkylpentylglycine and N-alkylalanine include lower alkyl groups preferably of 1 to about 6 carbon atoms, more preferably of 1 to 4 carbon atoms. Suitable compounds of formula (II) include those described in application Serial No. PCT/US98/24273, filed Nov. 13, 1998, entitled “Novel Exendin Agonist Compounds,” identified therein in Examples 1-89 (“Compounds 1-89,” respectively), as well as those corresponding compounds identified therein in Examples 104 and 105.

Preferred such exendin analogs include those wherein Xaa₁ is His, Ala or Norval. More preferably Xaa₁ is His or Ala. Most preferably Xaa₁ is His.

Preferred are those compounds of formula (IV) wherein Xaa₂ is Gly.

Preferred are those compounds of formula (IV) wherein Xaa₃ is Ala.

Preferred are those compounds of formula (IV) wherein Xaa₄ is Ala.

Preferred are those compounds of formula (IV) wherein Xaa₉ is Ala.

Preferred are those compounds of formula (IV) wherein Xaa₁₄ is Leu, pentylglycine or Met.

Preferred compounds of formula (IV) are those wherein Xaa₂₅ is Trp or Phe.

Preferred compounds of formula (IV) are those where Xaa₆ is Ala, Phe or naphthylalanine; Xaa₂₂ is Phe or naphthylalanine; and Xaa₂₃ is Ile or Val.

Preferred are compounds of formula (IV) wherein Xaa₃₁, Xaa₃₆, Xaa₃₇ and Xaa₃₈ are independently selected from Pro, homoproline, thioproline and N-alkylalanine.

Preferably Z₁ is —NH₂.

Preferably Z₂ is —NH₂.

According to one aspect, preferred are compounds of formula (IV) wherein Xaa₁ is Ala, His or Tyr, more preferably Ala or His; Xaa₂ is Ala or Gly; Xaa₆ is Phe or naphthylalanine; Xaa₁₄ is Ala, Leu, pentylglycine or Met; Xaa₂₂ is Phe or naphthylalanine; Xaa₂₃ is Ile or Val; Xaa₃₁, Xaa₃₆, Xaa₃₇ and Xaa₃₈ are independently selected from Pro, homoproline, thioproline or N-alkylalanine; and Xaa₃₉ is Ser or Tyr, more preferably Ser. More preferably Z₁ is —NH₂.

In an especially preferred aspect, preferred compounds include those of formula (IV) wherein: Xaa₁ is His or Ala; Xaa₂ is Gly or Ala; Xaa₃ is Ala, Asp or Glu; Xaa₄ is Ala or Gly; Xaa₅ is Ala or Thr; Xaa₆ is Phe or naphthylalanine; Xaa₇ is Thr or Ser; Xaa₈ is Ala, Ser or Thr; Xaa₉ is Ala, Asp or Glu; Xaa₁₀ is Ala, Leu or pentylglycine; Xaa₁₁ is Ala or Ser; Xaa₁₂ is Ala or Lys; Xaa₁₃ is Ala or Gln; Xaa₁₄ is Ala, Leu, Met or pentylglycine; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Phe or naphthylalanine; Xaa₂₃ is Ile, Val or tert-butylglycine; Xaa₂₄ is Ala, Glu or Asp; Xaa₂₅ is Ala, Trp or Phe; Xaa₂₆ is Ala or Leu; Xaa₂₇ is Ala or Lys; Xaa₂₈ is Ala or Asn; Z₁ is —OH, —NH₂, Gly-Z₂, Gly Gly-Z₂, Gly Gly Xaa₃₁-Z₂, Gly Gly Xaa₃₁ Ser-Z₂, Gly Gly Xaa₃₁ Ser Ser-Z₂, Gly Gly Xaa₃₁ Ser Ser Gly-Z₂, Gly Gly Xaa₃₁ Ser Ser Gly Ala-Z₂, Gly Gly Xaa₃₁ Ser Ser Gly Ala Xaa₃₆-Z₂, Gly Gly Xaa₃₁ Ser Ser Gly Ala Xaa₃₆ Xaa₃₇-Z₂, Gly Gly Xaa₃₁ Ser Ser Gly Ala Xaa₃₆ Xaa₃₇ Xaa₃₈-Z₂ or Gly Gly Xaa₃₁ Ser Ser Gly Ala Xaa₃₆ Xaa₃₇ Xaa₃₈ Xaa₃₉-Z₂; Xaa₃₁, Xaa₃₆, Xaa₃₇ and Xaa₃₈ being independently Pro homoproline, thioproline or N-methylalanine; and Z₂ being —OH or —NH₂; provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala; and provided also that, if Xaa₁ is His, Arg or Tyr, then at least one of Xaa₃, Xaa₄ and Xaa₉ is Ala. Especially preferred compounds of formula (IV) include those described in application Serial No. PCT/US98/24273, filed Nov. 13, 1998, entitled “Novel Exendin Agonist Compounds” as having the amino acid sequence of SEQ. ID. NOS. 5-93 therein.

According to an especially preferred aspect, provided are compounds of formula (IV) where Xaa₁₄ is Ala, Leu, Ile, Val or pentylglycine, more preferably Leu or pentylglycine, and Xaa₂₅ is Ala, Phe, Tyr or naphthylalanine, more preferably Phe or naphthylalanine. These compounds will be less susceptible to oxidative degration, both in vitro and in vivo, as well as during synthesis of the compound.

### Formula V

Also within the scope of the present invention are narrower genera of compounds having peptides of various lengths, for example genera of compounds which do not include peptides having a length of 28, 29 or 30 amino acid residues, respectively. Additionally, the present invention includes narrower genera of compounds described in PCT application Serial No. PCT/US98/24210, filed Nov. 13, 1998, entitled “Novel Exendin Agonist Compounds” and having particular amino acid sequences, for example, compounds of the formula (V) [SEQ. ID. NO:27]:

Xaa₁ Xaa₂ Xaa₃ Gly Xaa₅ Xaa₆ Xaa₇ Xaa₈ Xaa₉ Xaa₁₀
Xaa₁₁ Xaa₁₂ Xaa₁₃ Xaa₁₄ Xaa₁₅ Xaa₁₆ Xaa₁₇ Ala
Xaa₁₉ Xaa₂₀ Xaa₂₁ Xaa₂₂ Xaa₂₃ Xaa₂₄ Xaa₂₅ Xaa₂₆
Xaa₂₇ Xaa₂₈-Z₁;

wherein:

Xaa₁ is His or Arg;

Xaa₂ is Gly or Ala;

Xaa₃ is Asp or Glu;

Xaa₅ is Ala or Thr;

Xaa₆ is Ala, Phe or naphthylalanine;

Xaa₇ is Thr or Ser;

Xaa₈ is Ala, Ser or Thr;

Xaa₉ is Asp or Glu;

Xaa₁₀ is Ala, Leu or pentylglycine;

Xaa₁₁ is Ala or Ser;

Xaa₁₂ is Ala or Lys;

Xaa₁₃ is Ala or Gln;

Xaa₁₄ is Ala, Leu or pentylglycine;

Xaa₁₅ is Ala or Glu;

Xaa₁₆ is Ala or Glu;

Xaa₁₇ is Ala or Glu;

Xaa₁₉ is Ala or Val;

Xaa₂₀ is Ala or Arg;

Xaa₂₁ is Ala or Leu;

Xaa₂₂ is Phe or naphthylalanine;

Xaa₂₃ is Ile, Val or tert-butylglycine;

Xaa₂₄ is Ala, Glu or Asp;

Xaa₂₅ is Ala, Trp, or Phe;

Xaa₂₆ is Ala or Leu;

Xaa₂₇ is Ala or Lys;

Xaa₂₈ is Ala or Asn;

Z₁ is —OH,
—NH₂,
Gly-Z₂,
Gly Gly-Z₂,
Gly Gly Xaa₃₁-Z₂,
Gly Gly Xaa₃₁ Ser-Z₂,
Gly Gly Xaa₃₁ Ser Ser-Z₂,
Gly Gly Xaa₃₁ Ser Ser Gly-Z₂,
Gly Gly Xaa₃₁ Ser Ser Gly Ala-Z₂,
Gly Gly Xaa₃₁ Ser Ser Gly Ala Xaa₃₆-Z₂,
Gly Gly Xaa₃₁ Ser Ser Gly Ala Xaa₃₆ Xaa₃₇-Z₂ or
Gly Gly Xaa₃₁ Ser Ser Gly Ala Xaa₃₆ Xaa₃₇ Xaa₃₈-Z₂;
Xaa₃₁, Xaa₃₆, Xaa₃₇ and Xaa₃₈ are independently selected from the group consisting of Pro, homoproline, thioproline and N-methylylalanine; and
Z₂ is —OH or —NH₂;

provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala; and pharmaceutically acceptable salts thereof.

### Formula VI

Additionally, the present invention includes narrower genera of peptide compounds described in PCT Application Serial No. PCT/US98/24273, filed Nov. 13, 1998, entitled “Novel Exendin Agonist Compounds” as having particular amino acid sequences, for example, compounds of the formula [VI] [SEQ. ID. NO:28]:

Xaa₁ Xaa₂ Xaa₃ Xaa₅ Xaa₅ Xaa₆ Xaa₇ Xaa₈ Xaa₉ Xaa₁₀
Xaa₁₁ Xaa₁₂ Xaa₁₃ Xaa₁₄ Xaa₁₅ Xaa₁₆ Xaa₁₇ Ala
Xaa₁₉ Xaa₂₀ Xaa₂₁ Xaa₂₂ Xaa₂₃ Xaa₂₄ Xaa₂₅ Xaa₂₆
Xaa₂₇ Xaa₂₈-Z₁;

wherein:

Xaa₁ is His or Ala;

Xaa₂ is Gly or Ala;

Xaa₃ is Ala, Asp or Glu;

Xaa₄ is Ala or Gly;

Xaa₅ is Ala or Thr;

Xaa₆ is Phe or naphthylalanine;

Xaa₇ is Thr or Ser;

Xaa₈ is Ala, Ser or Thr;

Xaa₉ is Ala, Asp or Glu;

Xaa₁₀ is Ala, Leu or pentylglycine;

Xaa₁₁ is Ala or Ser;

Xaa₁₂ is Ala or Lys;

Xaa₁₃ is Ala or Gln;

Xaa₁₄ is Ala, Leu, Met or pentylglycine;

Xaa₁₅ is Ala or Glu;

Xaa₁₆ is Ala or Glu;

Xaa₁₇ is Ala or Glu;

Xaa₁₉ is Ala or Val;

Xaa₂₀ is Ala or Arg;

Xaa₂₁ is Ala or Leu;

Xaa₂₂ is Phe or naphthylalanine;

Xaa₂₃ is Ile, Val or tert-butylglycine;

Xaa₂₄ is Ala, Glu or Asp;

Xaa₂₅ is Ala, Trp or Phe;

Xaa₂₆ is Ala or Leu;

Xaa₂₇ is Ala or Lys;

Xaa₂₈ is Ala or Asn;

Z₁ is —OH,
—NH₂,
Gly-Z₂,
Gly Gly-Z₂
Gly Gly Xaa₃₁-Z₂,
Gly Gly Xaa₃₁ Ser-Z₂,
Gly Gly Xaa₃₁ Ser Ser-Z₂,
Gly Gly Xaa₃₁ Ser Ser Gly-Z₂,
Gly Gly Xaa₃₁ Ser Ser Gly Ala-Z₂,
Gly Gly Xaa₃₁ Ser Ser Gly Ala Xaa₃₆-Z₂,
Gly Gly Xaa₃₁ Ser Ser Gly Ala Xaa₃₆ Xaa₃₇-Z₂
Gly Gly Xaa₃₁ Ser Ser Gly Ala Xaa₃₆ Xaa₃₇ Xaa₃8-Z₂
Gly Gly Xaa₃₁ Ser Ser Gly Ala Xaa₃₆ Xaa₃₇ Xaa₃₈ Ser-Z₂;
Xaa₃₁, Xaa₃₆, Xaa₃₇ and Xaa₃₈ are independently Pro, homoproline, thioproline, or N-methylylalanine; and
Z₂ is —OH or —NH₂;

provided that no more than three of Xaa₃, Xaa₆, Xaa₇, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇, and Xaa₂8 are Ala; and provided that, if Xaa₁ is His, Arg or Tyr, then at least one of Xaa₃, Xaa₄ and Xaa₉ is Ala; and pharmaceutically acceptable salts thereof.

Preferred compounds of formula (VI) include those wherein Xaa₁ is His, Ala, Norval or 4-imidazopropionyl. Preferably, Xaa₁ is His, or 4-imidazopropionyl or Ala, more preferably His or 4-imidazopropionyl.

Preferred compounds of formula (VI) include those wherein Xaa₂ is Gly.

Preferred compounds of formula (VI) include those wherein Xaa₄ is Ala.

Preferred compounds of formula (VI) include those wherein Xaa₉ is Ala.

Preferred compounds of formula (VI) include those wherein Xaa₁₄ is Leu, pentylglycine or Met.

Preferred compounds of formula (VI) include those wherein Xaa₂₅ is Trp or Phe.

Preferred compounds of formula (VI) include those wherein Xaa₆ is Ala, Phe or naphthylalanine; Xaa₂₂ is Phe or naphthylalanine; and Xaa₂₃ is Ile or Val.

Preferred compounds of formula (VI) include those wherein Z₁ is —NH₂.

Preferred compounds of formula (VI) include those wherein Xaa₃₁, Xaa₃₆, Xaa₃₇ and Xaa₃₈ are independently selected from the group consisting of Pro, homoproline, thioproline and N-alkylalanine.

Preferred compounds of formula (VI) include those wherein Xaa₃₉ is Ser or Tyr, preferably Ser. Preferred compounds of formula (VI) include those wherein Z₂ is —NH₂.

Preferred compounds of formula (VI) include those 42 wherein Z₁ is —NH₂.

Preferred compounds of formula (VI) include those wherein Xaa₂₁ is Lys-NH₂—R where R is Lys, Arg, C₁-C₁₀ straight chain or branched alkanoyl.

Preferred compounds of formula (VI) include those wherein X1 is Lys Asn, Lys-NHε-R Asn, or Lys-NHε-R Ala where R is Lys, Arg, C1-C10 straight chain or branched alkanoyl. Preferred compounds of formula (VI) include those having an amino acid sequence described in PCT application Serial No. PCT/US98/24273, filed Nov. 13, 1998, entitled “Novel Exendin Agonist Compounds” as being selected from SEQ. ID. NOS. 95-110 therein.

### Formula VII

Also provided are compounds described in PCT application PCT/US98/24210, filed Nov. 13, 1998, entitled “Novel Exendin Agonist Compounds”, including compounds of the formula (VII) [SEQ. ID. NO. 29]:

Xaa₁ Xaa₂ Xaa₃ Gly Xaa₅ Xaa₆ Xaa₇ Xaa₈ Xaa₉ Xaa₁₀
Xaa₁₁ Xaa₁₂ Xaa₁₃ Xaa₁₄ Xaa₁₅ Xaa₁₆ Xaa₁₇ Ala
Xaa₁₉ Xaa₂₀ Xaa₂₁ Xaa₂₂ Xaa₂₃ Xaa₂₄ Xaa₂₅ Xaa₂₆
X₁-Z₁;

wherein

Xaa₁ is His, Arg or Tyr or 4-imidazopropionyl;

Xaa₂ is Ser, Gly, Ala or Thr;

Xaa₃ is Asp or Glu;

Xaa₅ is Ala or Thr;

Xaa₆ is Ala, Phe, Tyr or naphthylalanine;

Xaa₇ is Thr or Ser;

Xaa₈ is Ala, Ser or Thr;

Xaa₉ is Asp or Glu;

Xaa₁₀ is Ala, Leu, Ile, Val, pentylglycine or Met;

Xaa₁₁ is Ala or Ser;

Xaa₁₂ is Ala or Lys;

Xaa₁₃ is Ala or Gln;

Xaa₁₄ is Ala, Leu, Ile, pentylglycine, Val or Met;

Xaa₁₅ is Ala or Glu;

Xaa₁₆ is Ala or Glu;

Xaa₁₇ is Ala or Glu;

Xaa₁₉ is Ala or Val;

Xaa₂₀ is Ala or Arg;

Xaa₂₁ is Ala, Leu or Lys-NH^{ε}—R where R is Lys, Arg, C₁-C₁₀ straight chain or branched alkanoyl or cycloalkylalkanoyl;

Xaa₂₂ is Phe, Tyr or naphthylalanine;

Xaa₂₃ is Ile, Val, Leu, pentylglycine, tert-butylglycine or Met;

Xaa₂₄ is Ala, Glu or Asp;

Xaa₂₅ is Ala, Trp, Phe, Tyr or naphthylalanine;

Xaa₂₆ is Ala or Leu;

X₁ is Lys Asn, Asn Lys, Lys-NH^{ε}—R Asn, Asn Lys-NH^{ε}—R, Lys-NH^{ε}—R Ala, Ala Lys-NH^{ε}—R where R is Lys, Arg, C₁-C₁₀ straight chain or branched alkanoyl or cycloalkylalkanoyl

Z₁ is —OH,
—NH₂,
Gly-Z₂,
Gly Gly-Z₂,
Gly Gly Xaa₃₁-Z₂,
Gly Gly Xaa₃₁ Ser-Z₂,
Gly Gly Xaa₃₁ Ser Ser-Z₂,
Gly Gly Xaa₃₁ Ser Ser Gly-Z₂,
Gly Gly Xaa₃₁ Ser Ser Gly Ala-Z₂,
Gly Gly Xaa₃₁ Ser Ser Gly Ala Xaa₃₆-Z₂,
Gly Gly Xaa₃₁ Ser Ser Gly Ala Xaa₃₆ Xaa₃₇-Z₂ or
Gly Gly Xaa₃₁ Ser Ser Gly Ala Xaa₃₆ Xaa₃₇ Xaa₃₈-Z₂;
Xaa₃₁, Xaa₃₆, Xaa₃₇ and Xaa₃₈ are independently selected from the group consisting of Pro, homoproline, 3Hyp, 4Hyp, thioproline, N-alkylglycine, N-alkylpentylglycine and N-alkylalanine; and
Z₂ is —OH or —NH₂;

provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, and Xaa₂₆ are Ala. Also within the scope of the present invention are pharmaceutically acceptable salts of the compound of formula (VII) and pharmaceutical compositions including said compounds and salts thereof.

Preferred exendin analogs of formula (VII) include those wherein Xaa₁ is His, Tyr or 4-imidazopropionyl. More preferably Xaa₁ is His.

Preferred are those compounds of formula (VII) wherein Xaa₁ is 4-imidazopropionyl.

Preferred are those compounds of formula (VII) wherein Xaa₂ is Gly.

Preferred compounds of formula (VII) are those wherein Xaa₁₄ is Leu, pentylglycine or Met.

Preferred compounds of formula (VII) are those wherein Xaa₂₅ is Trp or Phe.

According to one aspect, preferred are compounds of formula (VII) wherein Xaa₆ is Phe or naphthylalanine; and Xaa₂₂ is Phe or naphthylalanine; and Xaa₂₃ is Ile or Val. More preferably, Z₁ is —NH₂. According to one aspect, especially preferred are such compounds of formula (VII) wherein Xaa₃₁, Xaa₃₆, Xaa₃₇ and Xaa₃₈ are independently selected from the group consisting of Pro, homoproline, thioproline and N-alkylalanine. More prefers, Z₂ is —NH₂.

Preferred compounds of formula (VII) include those wherein X₁ is Lys Asn, Lys-NH^{ε}—R Asn, or Lys-NH^{ε}—R Ala where R is Lys, Arg, C₁-C₁₀ straight chain or branched alkanoyl. Preferred compounds of formula (VII) include compounds described in PCT application Serial No. PCT/US98/24210, filed Nov. 13, 1998, entitled “Novel Exendin Agonist Compounds” and identified therein as Compound Nos. 62-69.

Preferred such exendin analogs include those wherein Xaa₁ is His, Ala or Norval. More preferably Xaa₁ is His or Ala. Most preferably Xaa₁ is His.

Preferred are those compounds of formula (VII) wherein Xaa₂ is Gly.

Preferred are those compounds of formula (VII) wherein Xaa₃ is Ala.

Preferred are those compounds of formula (VII) wherein Xaa₄ is Ala.

Preferred are those compounds of formula (VII) wherein Xaa₉ is Ala.

Preferred are those compounds of formula (VII) wherein Xaa₁₄ is Leu, pentylglycine or Met.

Preferred compounds of formula (VII) are those wherein Xaa₂₅ is Trp or Phe.

Preferred compounds of formula (VII) are those where Xaa₆ is Ala, Phe or naphthylalanine; Xaa₂₂ is Phe or naphthylalanine; and Xaa₂₃ is Ile or Val.

Preferred are compounds of formula (VII) wherein Xaa₃₁, Xaa₃₆, Xaa₃₇ and Xaa₃₈ are independently selected from Pro, homoproline, thioproline and N-alkylalanine.

Preferably Z₁ is —NH₂.

Preferably Z₂ is —NH₂.

According to one aspect, preferred are compounds of formula (VII) wherein Xaa₁ is Ala, His or Tyr, more preferably Ala or His; Xaa₂ is Ala or Gly; Xaa₆ is Phe or naphthylalanine; Xaa₁₄ is Ala, Leu, pentylglycine or Met; Xaa₂₂ is Phe or naphthylalanine; Xaa₂₃ is Ile or Val; Xaa₃₁, Xaa₃₆, Xaa₃₇ and Xaa₃₈ are independently selected from Pro, homoproline, thioproline or N-alkylalanine; and Xaa₃₉ is Ser or Tyr, more preferably Ser. More preferably Z₁ is —NH₂.

According to an especially preferred aspect, preferred compounds include those of formula (VII) wherein: Xaa₁ is His or Ala; Xaa₂ is Gly or Ala; Xaa₃ is Ala, Asp or Glu; Xaa₄ is Ala or Gly; Xaa₅ is Ala or Thr; Xaa₆ is Phe or naphthylalanine; Xaa₇ is Thr or Ser; Xaa₈ is Ala, Ser or Thr; Xaa₉ is Ala, Asp or Glu; Xaa₁₀ is Ala, Leu or pentylglycine; Xaa₁₁ is Ala or Ser; Xaa₁₂ is Ala or Lys; Xaa₁₃ is Ala or Gln; Xaa₁₄ is Ala, Leu, Met or pentylglycine; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Phe or naphthylalanine; Xaa₂₃ is Ile, Val or tert-butylglycine; Xaa₂₄ is Ala, Glu or Asp; Xaa₂₅ is Ala, Trp or Phe; Xaa₂₆ is Ala or Leu; Xaa₂₇ is Ala or Lys; Xaa₂₈ is Ala or Asn; Z₁ is —OH, —NH₂, Gly-Z₂, Gly Gly-Z₂, Gly Gly Xaa₃₁-Z₂, Gly Gly Xaa₃₁ Ser-Z₂, Gly Gly Xaa₃₁ Ser Ser-Z₂, Gly Gly Xaa₃₁ Ser Ser Gly-Z₂, Gly Gly Xaa₃₁ Ser Ser Gly Ala-Z₂, Gly Gly Xaa₃₁ Ser Ser Gly Ala Xaa₃₆-Z₂, Gly Gly Xaa₃₁ Ser Ser Gly Ala Xaa₃₆ Xaa₃₇-Z₂, Gly Gly Xaa₃₁ Ser Ser Gly Ala Xaa₃₆ Xaa₃₇ Xaa₃8-Z₂ or Gly Gly Xaa₃₁ Ser Ser Gly Ala Xaa₃₆ Xaa₃₇ Xaa₃₈ Xaa₃₉-Z₂; Xaa₃₁, Xaa₃₆, Xaa₃₇ and Xaa₃₈ being independently Pro homoproline, thioproline or N-methylalanine; and Z₂ being —OH or —NH₂; provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala; and provided also that, if Xaa₁ is His, Arg or Tyr, then at least one of Xaa₃, Xaa₄ and Xaa₉ is Ala. Especially preferred compounds of formula (VII) include those described in PCT application Serial No. PCT/US98/24210, filed Nov. 13, 1998, entitled “Novel Exendin Agonist Compounds” and having the amino acid sequences identified therein as SEQ. ID. NOS. 5-93.

According to an especially preferred aspect, provided are compounds of formula (VII) where Xaa₁₄ is Ala, Leu, Ile, Val or pentylglycine, more preferably Leu or pentylglycine, and Xaa₂₅ is Ala, Phe, Tyr or naphthylalanine, more preferably Phe or naphthylalanine. These compounds will be less susceptible to oxidative degration, both in vitro and in vivo, as well as during synthesis of the compound.

### Formula VIII

Also provided are peptide compounds described in PCT Application Serial No. PCT/US98/24273, filed Nov. 13, 1998, entitled “Novel Exendin Agonist Compounds”, including compounds of the formula (VIII) [SEQ. ID. NO:30]:

Xaa₁ Xaa₂ Xaa₃ Xaa₄ Xaa₅ Xaa₆ Xaa₇ Xaa₈ Xaa₉ Xaa₁₀
Xaa₁₁ Xaa₁₂ Xaa₁₃ Xaa₁₄ Xaa₁₅ Xaa₁₆ Xaa₁₇ Ala
Xaa₁₉ Xaa₂₀ Xaa₂₁ Xaa₂₂ Xaa₂₃ Xaa₂₄ Xaa₂₅ Xaa₂₆
X₁-Z₁;

wherein

Xaa₁ is His, Arg, Tyr, Ala, Norval, Val, Norleu or 4-imidazopropionyl;

Xaa₂ is Ser, Gly, Ala or Thr;

Xaa₃ is Ala, Asp or Glu;

Xaa₄ is Ala, Norval, Val, Norleu or Gly;

Xaa₅ is Ala or Thr;

Xaa₆ is Phe, Tyr or naphthylalanine;

Xaa₇ is Thr or Ser;

Xaa₈ is Ala, Ser or Thr;

Xaa₉ is Ala, Norval, Val, Norleu, Asp or Glu;

Xaa₁₀ is Ala, Leu, Ile, Val, pentylglycine or Met;

Xaa₁₁ is Ala or Ser;

Xaa₁₂ is Ala or Lys;

Xaa₁₃ is Ala or Gln;

Xaa₁₄ is Ala, Leu, Ile, pentylglycine, Val or Met;

Xaa₁₅ is Ala or Glu;

Xaa₁₆ is Ala or Glu;

Xaa₁₇ is Ala or Glu;

Xaa₁₉ is Ala or Val;

Xaa₂₀ is Ala or Arg;

Xaa₂₁ is Ala, Leu or Lys-NH^{ε}—R where R is Lys, Arg, C¹⁻¹⁰ straight chain or branched alkanoyl or cycloallyl-alkanoyl;

Xaa₂₂ is Phe, Tyr or naphthylalanine;

Xaa₂₃ is Ile, Val, Leu, pentylglycine, tert-butylglycine or Met;

Xaa₂₄ is Ala, Glu or Asp;

Xaa₂₅ is Ala, Trp, Phe, Tyr or naphthylalanine;

Xaa₂₆ is Ala or Leu;

X₁ is Lys Asn, Asn Lys, Lys-NH^{ε}—R Asn, Asn Lys-NH^{ε}—R, Lys-NH^{ε}—R Ala, Ala Lys-NH^{ε}—R where R is Lys, Arg, C₁-C₁₀ straight chain or branched alkanoyl or cycloalkylalkanoyl

Z₁ is —OH,
—NH₂,
Gly-Z₂,
Gly Gly-Z₂,
Gly Gly Xaa₃′-Z₂,
Gly Gly Xaa₃₁ Ser-Z₂,
Gly Gly Xaa₃₁ Ser Ser-Z₂,
Gly Gly Xaa₃₁ Ser Ser Gly-Z₂,
Gly Gly Xaa₃₁ Ser Ser Gly Ala-Z₂,
Gly Gly Xaa₃₁ Ser Ser Gly Ala Xaa₃₆-Z₂,
Gly Gly Xaa₃₁ Ser Ser Gly Ala Xaa₃₆ Xaa₃₇-Z₂,
Gly Gly Xaa₃₁ Ser Ser Gly Ala Xaa₃₆ Xaa₃₇ Xaa₃₈-Z₂ or
Gly Gly Xaa₃₁ Ser Ser Gly Ala Xaa₃₆ Xaa₃₇ Xaa₃₈ Xaa₃₉-Z₂;
Xaa₃₁, Xaa₃₆, Xaa₃₇ and Xaa₃₈ are independently selected from the group consisting of Pro, homoproline, 3Hyp, 4Hyp, thioproline, N-alkylglycine, N-alkylpentylglycine and N-alkylalanine; and
Z₂ is —OH or —NH₂;

provided that no more than three of Xaa₃, Xaa₄, Xaa₅, Xaa₆, Xaa₈, Xaa₉, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, are Ala; and provided also that, if Xaa₁ is His, Arg, Tyr, or 4-imidazopropionyl then at least one of Xaa₃, Xaa₄ and Xaa₉ is Ala.

Preferred compounds of formula (VIII) include those wherein Xaa₁ is His, Ala, Norval or 4-imidazopropionyl. Preferably, Xaa₁ is His, or 4-imidazopropionyl or Ala, more preferably His or 4-imidazopropionyl.

Preferred compounds of formula (VIII) include those wherein Xaa₂ is Gly.

Preferred compounds of formula (VIII) include those wherein Xaa₄ is Ala.

Preferred compounds of formula (VIII) include those wherein Xaa₉ is Ala.

Preferred compounds of formula (VIII) include those wherein Xaa₁₄ is Leu, pentylglycine or Met.

Preferred compounds of formula (VIII) include those wherein Xaa₂₅ is Trp or Phe.

Preferred compounds of formula (VIII) include those wherein Xaa₆ is Ala, Phe or naphthylalanine; Xaa₂₂ is Phe or naphthylalanine; and Xaa₂₃ is Ile or Val.

Preferred compounds of formula (VIII) include those wherein Z₁ is —NH₂.

Preferred compounds of formula (VIII) include those wherein Xaa₃₁, Xaa₃₆, Xaa₃₇ and Xaa₃₈ are independently selected from the group consisting of Pro, homoproline, thioproline and N-alkylalanine.

Preferred compounds of formula (VIII) include those wherein Xaa₃₉ is Ser or Tyr, preferably Ser. Preferred compounds of formula (VIII) include those wherein Z₂ is —NH₂.

Preferred compounds of formula (VIII) include those 42 wherein Z₁ is —NH₂.

Preferred compounds of formula (VIII) include those wherein Xaa₂₁ is Lys-NH^{ε}—R where R is Lys, Arg, C₁-C₁₀ straight chain or branched alkanoyl.

Preferred compounds of formula (VIII) include those wherein X₁ is Lys Asn, Lys-NH^{ε}—R Asn, or Lys-NH^{ε}—R Ala where R is Lys, Arg, C₁-C₁₀ straight chain or branched alkanoyl.

Preferred compounds of formula (VIII) include those described in PCT Application Serial No. PCT/US98/24273, filed Nov. 13, 1998, entitled “Novel Exendin Agonist Compounds” as having an amino acid sequence selected from those identified therein as SEQ. ID. NOS. 95-110.

### Formula IX

Compounds particularly useful according to the present invention are exendin analogs with agonist activity described in U.S. patent application Ser. No. 09/003,869, filed Jan. 7, 1998, entitled “Use of Exendins And Agonists Thereof For The Reduction of Food Intake”, including compounds of the formula (IX) [SEQ. ID. NO:31]:

Xaa₁ Xaa₂ Xaa₃ Gly Thr Xaa₄ Xaa₅ Xaa₆ Xaa₇ Xaa₈
Ser Lys Gln Xaa₉ Glu Glu Glu Ala Val Arg Leu
Xaa₁₀ Xaa₁₁ Xaa₁₂ Xaa₁₃ Leu Lys Asn Gly Gly Xaa₁₄
Ser Ser Gly Ala Xaa₁₅ Xaa₁₆ Xaa₁₇ Xaa₁₈-Z

wherein:

Xaa₁ is His, Arg or Tyr;

Xaa₂ is Ser, Gly, Ala or Thr;

Xaa₃ is Asp or Glu;

Xaa₄ is Phe, Tyr or naphthalanine;

Xaa₅ is Thr or Ser;

Xaa₆ is Ser or Thr;

Xaa₇ is Asp or Glu;

Xaa₈ is Leu, Ile, Val, pentylglycine or Met;

Xaa₉ is Leu, Ile, pentylglycine, Val or Met;

Xaa₁₀ is Phe, Tyr or naphthalanine;

Xaa₁ is Ile, Val, Leu, pentylglycine, tert-butylglycine or Met;

Xaa₁₂ is Glu or Asp; Xaa₁₃ is Trp, Phe, Tyr, or naphthylalanine;

Xaa₁₄, Xaa₁₅, Xaa₁₆ and Xaa₁₇ are independently Pro, homoproline, 3Hyp, 4Hyp, thioproline, N-alkylglycine, N-alkylpentylglycine or N-alkylalanine;

Xaa₁₈ is Ser, Thr or Tyr; and Z is —OH or —NH₂;

with the proviso that the compound does not have the formula of either SEQ. ID. NOS:12 or 14. Preferred N-alkyl groups for N-alkylglycine, N-alkylpentylglycine and N-alkylalanine include lower alkyl groups preferably of 1 to about 6 carbon atoms, more preferably of 1 to 4 carbon atoms. Also useful in the present invention are pharmaceutically acceptable salts of the compounds of formula (IX).

Preferred exendin analogs include those wherein Xaa₁ is His or Tyr. More preferably Xaa₁ is His.

Preferred are those compounds wherein Xaa₂ is Gly.

Preferred are those compounds wherein Xaa₉ is Leu, pentylglycine or Met.

Preferred compounds include those wherein Xaa₁₃ is Trp or Phe.

Also preferred are compounds where Xaa₄ is Phe or naphthalanine; Xaa₁₁ is Ile or Val and Xaa₁₄, Xaa₁₅, Xaa₁₆ and Xaa₁₇ are independently selected from Pro, homoproline, thioproline or N-alkylalanine. Preferably N-alkylalanine has a N-alkyl group of 1 to about 6 carbon atoms.

According to an especially preferred aspect, Xaa₁₅, Xaa₁₆ and Xaa₁₇ are the same amino acid reside.

Preferred are compounds wherein Xaa₁₈ is Ser or Tyr, more preferably Ser. Preferably Z is —NH₂.

According to one aspect, preferred are compounds of formula (VII) wherein Xaa₁ is His or Tyr, more preferably His; Xaa₂ is Gly; Xaa₄ is Phe or naphthalanine; Xaa₉ is Leu, pentylglycine or Met; Xaa₁₀ is Phe or naphthalanine; Xaa₁₁ is Ile or Val; Xaa₁₄, Xaa₁₅, Xaa₁₆ and Xaa₁₇ are independently selected from Pro, homoproline, thioproline or N-alkylalanine; and Xaa₁₈ is Ser or Tyr, more preferably Ser. More preferably Z is —NH₂.

According to an especially preferred aspect, especially preferred compounds include those of formula (IX) wherein: Xaa₁ is His or Arg; Xaa₂ is Gly; Xaa₃ is Asp or Glu; Xaa₄ is Phe or naphthylalanine; Xaa₅ is Thr or Ser; Xaa₆ is Ser or Thr; Xaa₇ is Asp or Glu; Xaa₈ is Leu or pentylglycine; Xaa₉ is Leu or pentylglycine; Xaa₁₀ is Phe or naphthylalanine; Xaa₁ is Ile, Val or t-butyltylglycine; Xaa₁₂ is Glu or Asp; Xaa₁₃ is Trp or Phe; Xaa₁₄, Xaa₁₅, Xaa₁₆, and Xaa₁₇ are independently Pro, homoproline, thioproline, or N-methylalanine; Xaa₁₈ is Ser or Tyr: and Z is —OH or —NH₂; with the proviso that the compound does not have the formula of either SEQ. ID. NOS. 7 or 9. More preferably Z is —NH₂.

According to an especially preferred aspect, provided are compounds where Xaa₉ is Leu, Ile, Val or pentylglycine, more preferably Leu or pentylglycine, and Xaa₁₃ is Phe, Tyr or naphthylalanine, more preferably Phe or naphthylalanine. These compounds are believed to exhibit advantageous duration of action and to be less subject to oxidative degration, both in vitro and in vivo, as well as during synthesis of the compound.

### Formula X

Also provided are compounds described in PCT Application Serial No. PCT/US98/16387, filed Aug. 6, 1998, entitled “Novel Exendin Agonist Compounds”, including compounds of the formula (X) [SEQ. ID. NO:32]:

Xaa₁ Xaa₂ Xaa₃ Gly Thr Xaa₄ Xaa₅ Xaa₆ Xaa₇ Xaa₈
Ser Lys Gln Xaa₉ Glu Glu Glu Ala Val Arg Leu
Xaa₁₀ Xaa₁₁ Xaa₁₂ Xaa₁₃ Leu X₁ Gly Gly Xaa₁₄
Ser Ser Gly Ala Xaa₁₅ Xaa₁₆ Xaa₁₇ Xaa₁₈-Z

wherein:

Xaa₁ is His, Arg, Tyr or 4-imidazopropionyl;

Xaa₂ is Ser, Gly, Ala or Thr;

Xaa₃ is Asp or Glu;

Xaa₄ is Phe, Tyr or naphthylalanine;

Xaa₅ is Thr or Ser;

Xaa₆ is Ser or Thr;

Xaa₇ is Asp or Glu;

Xaa₈ is Leu, Ile, Val, pentylglycine or Met;

Xaa₉ is Leu, Ile, pentylglycine, Val or Met;

Xaa₁₀ is Phe, Tyr or naphthylalanine;

Xaa₁₁ is Ile, Val, Leu, pentylglycine, tert-butylglycine or Met;

Xaa₁₂ is Glu or Asp;

Xaa₁₃ is Trp, Phe, Tyr, or naphthylalanine; X₁ is Lys Asn, Asn Lys, Lys-NH—R Asn, Asn Lys-NH—R where R is Lys, Arg, C₁-C₁₀ straight chain or branched alkanoyl or cycloalkylalkanoyl;

Xaa₁₄, Xaa₁₅, Xaa₁₆ and Xaa₁₇ are independently Pro, homoproline, 3Hyp, 4Hyp, thioproline, N-alkylglycine, N-alkylpentylglycine or N-alkylalanine;

Xaa₁₈ is Ser, Thr or Tyr; and Z is —OH or —NH₂;

with the proviso that the compound does not have the formula of either SEQ. ID. NOS. 7 or 9. Suitable compounds of formula (X) include compounds described in PCT Application Serial No. PCT/US98/16387, filed Aug. 6, 1998, entitled “Novel Exendin Agonist Compounds” having the amino acid sequences of SEQ. ID. NOS. 37-40 therein.

Preferred exendin analogs of formula (X) include those wherein Xaa₁ is His, Tyr or 4-imidazopropionyl. More preferably, Xaa₁ is His or 4-imidazopropionyl.

Preferred are those compounds of formula (X) wherein Xaa₂ is Gly.

Preferred are those compounds of formula (X) wherein Xaa₉ is Leu, pentylglycine or Met.

Preferred are those compounds of formula (X) wherein Xaa₁₃ is Trp or Phe.

Preferred are those compounds of formula (X) wherein

X₁ is Lys Asn, or Lys-NH^{ε}—R Asn, where R is Lys, Arg, C₁-C₁₀ straight chain or branched alkanoyl.

Also preferred are compounds of formula (X) wherein Xaa₄ is Phe or naphthylalanine; Xaa₁₀ is Phe or naphthylalanine; Xaa₁ is Ile or Val and Xaa₁₄, Xaa₁₅, Xaa₁₆ and Xaa₁₇ are independently selected from Pro, homoproline, thioproline or N-alkylalanine. According to an especially preferred aspect, Xaa₁₈ is Ser or Tyr. Preferred are those such compounds wherein Xaa₁₈ is Ser. Preferably, Z is —NH₂.

According to one preferred aspect, preferred are compounds of formula (X) wherein Xaa₄ is Phe or naphthylalanine; Xaa₁₀ is Phe or naphthylalanine; Xaa₁₁ is Ile or Val, X₁ is Lys Asn, or Lys-NH^{ε}—R Asn, where R is Lys, Arg, C₁-C₁₀ straight chain or branched alkanoyl and Xaa₁₄, Xaa₁₅, Xaa₁₆ and Xaa₁₇ are independently selected from Pro, homoproline, thioproline or N-alkylalanine.

Exendins and exendin agonists that are peptides, such as exendin analogs, described herein may be prepared through peptide purification as described in, for example, Eng, et al., J. Biol. Chem. 265:20259-62, 1990; and Eng, et al., J. Biol. Chem. 267:7402-05, 1992, hereby incorporated by reference herein. Alternatively, exendins, incretins, GLP-1s, and agonists, analogs, derivatives and variants that are peptides may be prepared by methods known to those skilled in the art, for example, as described in Raufmnan, et al., J. Biol. Chem. 267:21432-37, 1992), hereby incorporated by reference herein, using standard solid-phase peptide synthesis techniques and preferably an automated or semiautomated peptide synthesizer as previously described and is well known in the art.

Peptide molecules of the invention may also be prepared using recombinant DNA techniques, using methods now known in the art. See, e.g., Sambrook et al., Molecular Cloning: A Laboratory Manual, 2d Ed., Cold Spring Harbor (1989), with any necessary chemical modifications made to the molecules in additional steps as known in the art. Alternatively, such compounds may be prepared by homogeneous phase peptide synthesis methods. Non-peptide compounds useful in the present invention may be prepared by art-known methods. For example, phosphate-containing amino acids and peptides containing such amino acids, may be prepared using methods known in the art. See, e.g., Bartlett and Landen, Biorg. Chem. 14:356-377 (1986). Methods for making and/or purifying GLP-1 and its agonists, analogs, derivatives, variants, and fragments, as discussed previously, can also be utilized to make and/or purify exendins, their agonists, analogs, derivatives, variants, and fragments thereof.

Also included in the present invention are peptide sequences having greater than 50% or 55% amino acid sequence identity, and preferably greater than 70, 80, 90, or 95% amino acid sequence identity to SEQ ID NOs: 1, 12, and 14, as well as truncated sequences thereof. As used herein, sequence identity refers to a comparison made between two molecules using standard algorithms well known in the art. The preferred algorithm for calculating sequence identity for the present invention is the Smith-Waterman algorithm, for example, SEQ ID NO: 1 [i.e., GLP-1(1-37)], SEQ ID NO: 12 or 14 [exendin-3 and 4, respectively] can be used as the reference sequences to define the percentage identity of homology over their length. The choice of parameter values for matches, mismatches, and insertions or deletions is arbitrary, although some parameter values have been found to yield more biologically realistic results than others. One preferred set of parameter values for the Smith-Waterman algorithm is set forth in the “maximum similarity segments” approach, which uses values of 1 for a matched residue and −⅓ for a mismatched residue (a residue being either a single nucleotide or single amino acid). Waterman, Bull. Math. Biol. 46; 473 (1984). Insertions and deletions (indels), x, are weighted as xₖ=1+⅓k, where k is the number of residues in a given insert or deletion. Id.

For instance, a sequence that is identical to the 37-amino acid residue sequence of SEQ ID NO: 1, except for 18 amino acid substitutions and an insertion of 3 amino acids, would have a percent identity given by:

[(1×37 matches)−(⅓×18 mismatches)−(1+ 3/3 indels)]/37=78% “identity.”

This algorithm can be used with any amino acid sequence to determine sequence homology. For purposes of determining homology, truncation of the mature sequence should be disregarded. Sequences having lesser degrees of homology, comparable bioactivity, and equivalent expression characteristics are considered equivalents.

The biological activity of a GLP-1 agonist and/or analog can be determined by in vitro and in vivo animal models and human studies, as is well known to the skilled artisan. GLP-1 biological activity can be determined by standard methods, in general, by receptor binding activity screening procedures, which involve providing appropriate cells that express the GLP-1 receptor on their surface, for example, insulinoma cell lines such as RINmSF cells or INS-1 cells. See Mojsov, Int. J. Peptide Protein Res. 40; 333 (1992) and EP 0708179 A2. GLP-1 receptors are cell-surface proteins found, for example, on insulin-producing pancreatic β-cells; the GLP-1(7-36) receptor has been characterised in the art. Additional receptors at which GLP-1 and exendins act are also thought to exist, and may mediate effects by which the instant invention is operative. Methods of determining whether a chemical or peptide binds to or activates a particular GLP-1 receptor are known to the skilled artisan. For example, U.S. Pat. Nos. 6,051,689, 5,846,747, and 5,670,360 describe GLP-1 receptors, as well as methods for using them. Cells that are engineered to express a GLP-1 receptor also can be used. In addition to measuring specific binding of tracer to membrane using radioimmunoassay methods, cAMP activity or glucose dependent insulin production can also be measured. In one method, a polynucleotide encoding a GLP-1 receptor is employed to transfect cells so that they express the GLP-1 receptor protein. Thus, for example, these methods may be employed for screening for a receptor agonist by contacting such cells with compounds to be screened and determining whether such compounds generate a signal (i.e., activate the receptor). Other screening techniques include the use of cells that express the GLP-1 receptor, for example, transfected CHO cells, in a system to measure extracellular pH or ionic changes caused by receptor activation. For example, potential agonists may be contacted with a cell that expresses the GLP-1 protein receptor and a second messenger response (e.g., signal transduction or ionic or pH changes), may be measured to determine whether the potential agonist is effective.

The molecules of the present invention may be used in combination with a suitable pharmaceutical carrier. Such compositions comprise a therapeutically effective amount of the polypeptide, and a pharmaceutically acceptable carrier or excipient. The compounds of this invention can be administered in any effectively pharmaceutically acceptable form to animals, including human subjects, e.g. in topical, lavage, oral, suppository, parenteral, injectible and/or infusible dosage forms, as a topical, buccal, sublingual, pulmonary or nasal spray, or in any other manner effective to deliver the agents. The route of administration will preferably be designed to optimize delivery and/or localization of the agents, and for peptide molecules of the invention, is preferably via a subcutaneous or other parenteral injection route, or transmucosal delivery.

In addition to administration with conventional carriers, active ingredients may be administered by a variety of specialized delivery drug techniques which are known to those of skill in the art, such as portable infusion pumps.

Suitable formulations for the peptide molecules of the invention are disclosed in U.S. Ser. No. 09/899,330 and related applications, all of which are herein incorporated by reference. Additional formulations for administration may be made in accordance with methods and amounts known in the art, such as set forth in Remington's Pharmaceutical Sciences, 18th Ed., Wiley Publishing (1990), the disclosure of which is herein incorporated by references in its entirety.

The peptides of the present invention are administered along with a pharmaceutically acceptable carrier in an amount sufficient to prevent or treat nephropathy. The compounds of this invention have extremely low toxicity and a low degree of side effects even at high doses. The dosing range of the compounds of this invention will vary depending on a number of factors, such as route and manner of administration, i.e. sustained release or continuous, such as intravenous infusion or subcutaneous infusion, desired dosing schedule, etc.

Although not limited to the following ranges and provided only as an illustration, exemplary dose ranges for peptides of the invention can include 0.001 pmol/kg to 500 nmol/kg per day depending on the composition selected. A lower limit of a dosage range can be about 0.001 pmol/kg, 0.01 pmol/kg, 0.1 pmol/kg, 1 pmol/kg, 10 pmol/kg, or 100 pmol/kg. An upper dosage range can be about 10 pmol/kg, 100 pmol/kg, 1 nmol/kg, 10 nmol/kg, 100 nmol/kg, 250 nmol/kg or 500 nmol/kg. The desired dose will vary depending on the selected active composition and its relative potency compared to e.g., GLP-1 and exendin. The desired dose will also depend upon other factors including bioavailability, the route of administration and the formulation. For example, continuous infusion as well as bolus doses and sustained release formulations are contemplated and may include administration of the peptide in liquid, gel, semi-solid or solid form.

Alternatively, doses from about 0.0005 μg/kg/dose to about 12000 μg/kg/dose, depending on mode of administration, can be used to achieve therapeutic plasma levels (at least 5 pg/ml, preferably at least 40 pg/ml). For molecules having potency similar to exendin-4, preferably peak plasma levels will not exceed about 500 pg/ml, more preferably about 250 pg/ml, and most preferably about 150 pg/ml. Administered parenterally, exendins and agonists in an amount from about 0.001 μg/kg/dose to about 1.0 μg/kg/dose produce therapeutic effects.

Exemplary doses for continuous infusion by intravenous (I.V.) can be about 0.1 pmol/kg/min to 10 pmol/kg/min and by subcutaneous (s.c.) about 0.1 pmol/kg/min to 75 pmol/kg/min., and for single injection (bolus) by I.V. about 0.1 nmol/kg to 2.0 nmol/kg and s.c. about 0.1 nmol/kg to 100 nmol/kg. The foregoing doses may be administered as a single dose per day or may be divided into multiple doses for administration per day. The peptides of this invention may be administered once to several times daily.

While a preferred method of administration of a GLP-1 peptide may be through a continuous application, other forms of delivery as described above are also contemplated. However, an exemplary dosing rate can be within a range of from about 1 to about 10 pmol/kg per minute of GLP-1 delivered by sustained release subcutaneous, intramuscular, interperitoneal, injected depot with sustained release, deep lung insufflation, as well as by intravenous, buccal, patch or other sustained release delivery methods. Degradation-resistant GLP-1 analogs, derivatives or variants, exendins, analogs, derivatives or variants, and other molecules of the invention need not be delivered continuously, but are suitable for bolus or sustained release dosing and may be at doses much lower than those described.

Other drugs besides compositions of the invention which are compatible with the carrier ingredients may also be incorporated into the pharmaceutical formulations. Such drugs may be readily ascertained by those of ordinary skill in the art and may include, for instance, anti-inflammatory agents, diuretics, vasodilators, etc.

It is understood that the present invention contemplates the use of not only the above-stated active forms of the compositions of the invention, but also includes the prodrugs (proforms) which metabolize to the compound and biologically active salt forms thereof, as well as optical isomers which provide the same pharmaceutical results.

The compositions of the invention may also be used in combination with agents known in the art that enhance the half-life in vivo of peptide in order to enhance or prolong the biological activity of the peptide. For example, a molecule or chemical moiety may be covalently linked to the composition of the present invention before administration thereof. Alternatively, the enhancing agent may be administered concurrently with the composition. Still further, the agent may comprise a molecule that is known to inhibit the enzymatic degradation of the compositions of the invention that may be administered concurrently with or after administration of the composition. Such a molecule may be administered, for example, orally, by injection, or any other means known in the art.

While there is no hard and fast rule as to when or how often GLP-1 must be administered in accordance with this invention to prevent nephropathy, as a general guideline GLP-1 may be administered to a patient that has two or more risk factors present for developing the disease, including but not limited to insulin resistance, diabetes, history of uncontrolled high blood pressure, kidney disease, increased creatinine clearance level, proteinuria, and non-Caucasian racial decent.

The following examples are provided as illustrations of the utility of the peptide molecules of the invention, and are not intended to be limiting.

### EXAMPLES

Dahl S rats are insulin-resistant and rapidly develop severe hypertension and renal injury when fed a high salt diet. The increase in mean arterial pressure (MAP) is associated with sodium retention that can be prevented by servocontrolling total body fluid volume or by using diuretics. Dahl S rats exhibit many phenotypic traits associated with salt-sensitive hypertension in man. Specifically, they are salt-sensitive, insulin-resistant and hyperlipidemic. They also develop glomerulosclerosis following the development of hypertension. The type of renal injury seen in Dahl S rats fed a high salt diet resembles that seen in patients with diabetic nephropathy, and in hypertensive African-Americans, in whom the incidence of end-stage renal disease is 16 times higher than that seen in Caucasian hypertensive patients.

### Example 1

### Methods

Experiments were performed on male Dahl sensitive/Jr (Dahl S) rats maintained on a low salt diet (0.1% NaCl) from birth to prevent the development of hypertension. When the rats were 9 weeks old, they were anesthetized with an i.m. injection of ketamine (40 mg/kg), xylazine (2.5 mg/kg), and acepromazine (0.6 mg/kg) and catheters were implanted in the femoral artery and vein for chronic measurement of MAP (mean arterial pressure) and i.v. infusion (10 ml/day). The rats received an i.m. injection of enrofloxacin (Baytril, 2.5 mg/kg) to prevent infections and were given 4-5 days to recover from surgery.

Evaluation of Effects of rGLP-1 on MAP and Renal Dysfunction

MAP was measured on 3 consecutive days and a blood sample and an overnight urine sample was collected during the control period while the rats were maintained on a low salt diet (0.4 NaCl) and infused with the vehicle for recombinant glucagon-like peptide-1(7-36)amide (rGLP-1) (5% mannitol solution on 0.9% saline) at a rate of 10 ml/day. The rats were then switched to a high salt diet (8% NaCl) for 14 days. One group of rats received a continuous i.v. infusion of RGLP-1 at a dose of 1 ug/kg/min, while the other group of rats was infused with vehicle. MAP was directly recorded from the catheter implanted in the femoral artery on days 3, 7, 10 and 14 of the high salt diet using a computerized data acquisition system (WINDAQ software, DataQ Instruments Inc. Akron, Ohio) at a sample rate of 300 Hz between 11:00 AM and 3:00 PM while the rats were conscious and freely moving in their home cages. MAP was averaged over 1-min periods and converted to a mean value for the recording session. In addition, a blood sample was collected from the arterial catheter for measurement of the plasma creatinine concentration and an overnight urine sample was collected for measurement of proteinuria and microalbuminuria on days 7 and 14 after starting the high salt diet. Urine protein concentration was determined by the Bradford method (Bio-Rad Laboratories Hercules, Calif.) with bovine serum albumin as the standard. Urine albumin concentration was measured using the albumin blue 580 method (Molecular Probes, Eugene, Oreg.).

Histological Evaluation of the Kidney

At the end of the experiment, the rats were anesthetized with pentobarbital (60 mg/Kg, i.p.), and the kidney was collected, weighed and fixed in a 5% buffered formalin solution. The tissues were later embedded in paraffin, sectioned and stained with Mason's trichrome stain and examined by light microscopy. The degree of glomerulosclerosis was scored as previously described by Raij et al on a scale of 0-4 based on the percentage of glomerular capillary area filled with mesangial matrix. A score of 0 indicates no damage, a score of 2 indicates that 50% of the glomerular capillaries area is filled with matrix, and a score of 4 indicates complete closure of all capillaries within a given glomerulus. The kidney sections were also examined for the degree of renal interstitial fibrosis and the percentage of medullary area occupied by protein casts was determined using a Metamorph imaging program on at least 10 regions per kidney section.

Statistical Analysis

Mean values±SE are presented. The significance of differences in mean values measured in the vehicle- and RGLP-1 treated groups were analyzed using a two-way ANOVA for repeated measurements followed by the Duncan's multiple-range test or an unpaired t-test. The significance of differences within the group was tested using an ANOVA for repeated measures. A P value<0.05 was considered statistically significant.

### Example 2

### Effect of RGLP-1 on the Development of Hypertension

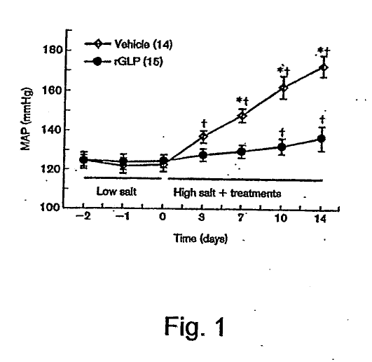

Rats were maintained on a low salt diet (0.4% NaCl) during the 3 day control period. The rats were then switched to a high salt diet (8% NaCl) and received either rGLP-1(1 μg/kg/min) or vehicle. FIG. 1 indicates the development of hypertension in vehicle-treated rats upon initiation of a high salt diet, and the protection afforded by administration of GLP-1. Numbers in parentheses indicate the number of rats studied. * indicates P<0.05 versus vehicle treatment and +indicates P<0.05 versus control value measured on a low salt diet. Baseline MAP measured while the rats were fed a low salt diet was similar in the rats subsequently treated with rGLP-1 or vehicle and averaged 122±2 mmHg. MAP increased to 174±6 mmHg in the vehicle-treated Dahl S rats fed a high salt diet for 14 days. In contrast, the rise in MAP was significantly attenuated and MAP only rose to 136±7 mmHg in the rats infused with rGLP-1.

### Example 3

### Effect of RGLP-1 on Renal Dysfunction

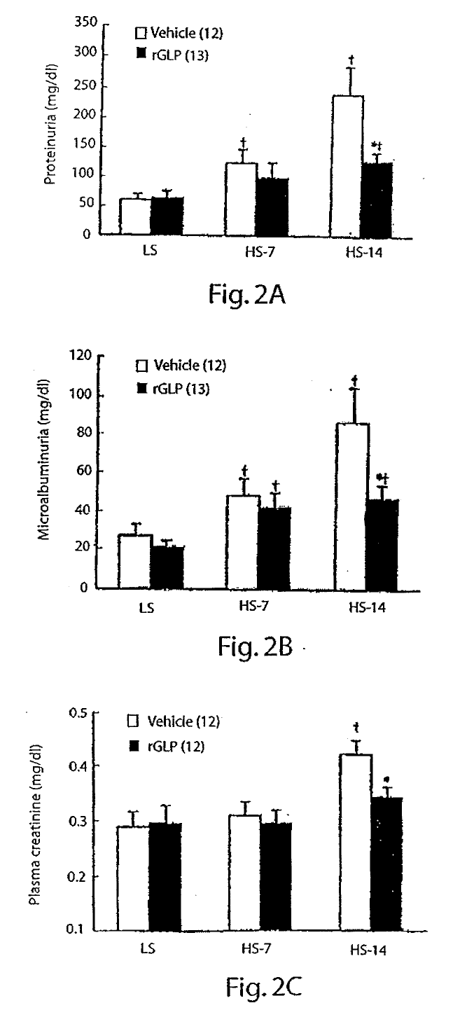

The effects of rGLP-1 on the development of proteinuria, microalbuminuria and plasma creatinine concentration (indicators of renal damage and nephropathy) in Dahl S rats fed a high salt diet are presented in FIG. 2. Rats were maintained on a low salt diet (0.4% NaCl) during the 3 day control period. The rats were then switched to a high salt diet (8% NaCl) and received either rGLP-1(1 μg/kg/min) or vehicle. LS: low salt diet, HS-7 or -14: 7 or 14 days after high salt diet. Numbers in parentheses indicate the number of rats studied. * indicates P<0.05 versus vehicle treatment and +indicates P<0.05 versus control value measured on a low salt diet. The excretion of protein and albumin increased significantly after 14 days on a high salt diet in Dahl S rats treated with vehicle (FIGS. 2A and 2B). This was associated with a significant increase in plasma creatinine concentration, an index of glomerular filtration rate (GFR, FIG. 2C). Chronic administration of rGLP-1 significantly attenuated the increase in urinary excretion of protein and albumin by 62% and 68%, respectively (FIGS. 2A and 2B). rGLP-1 also reduced the rise in plasma creatinine concentration in Dahl S rats fed a high salt diet by 62% (FIG. 2C).

### Example 4

### Reduction in Renal End-Organ Damage

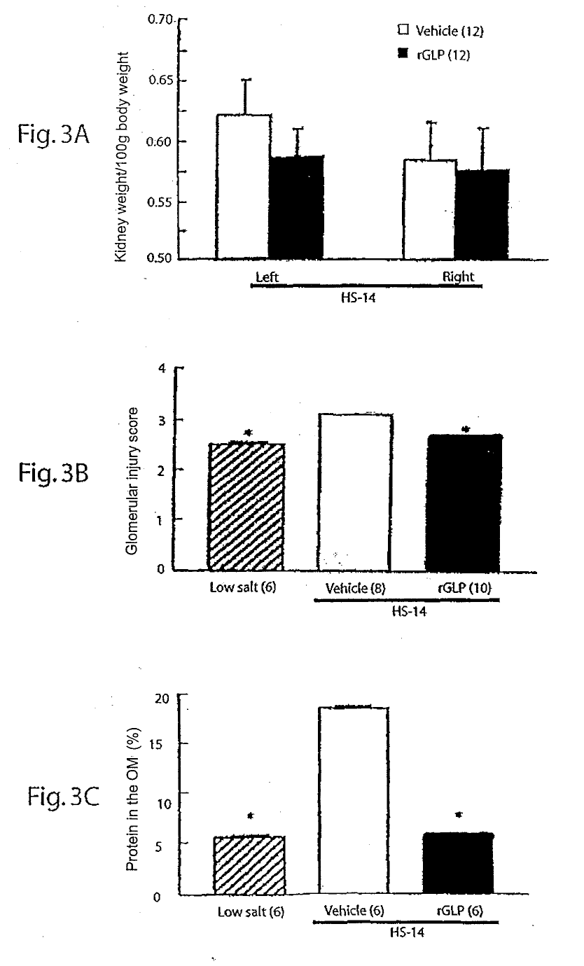

The effects of rGLP-1 on hypertension-induced renal end organ damage in Dahl S rats fed a high salt diet for 14 days (HS-14) is presented in FIG. 3. Rats were treated with RGLP-1 or vehicle. Low salt (LS), in FIGS. 3B and 3C, indicates results obtained from a separate group of normotensive Dahl S rats maintained on a low salt (0.4% NaCl) diet throughout the study. Numbers in parentheses indicate the number of rats studied. * indicates P<0.05 versus control Dahl S rats fed a high salt diet and treated with vehicle. Chronic treatment of Dahl S rats with RGLP-1 reduced the kidney weight (FIG. 3A), an index of renal hypertrophy. In vehicle-treated Dahl S rats there was marked expansion of the mesangial matrix in nearly every glomerulus examined and the overall glomerular injury score averaged 3.1, indicating that more than 75% of the area of glomerular capillaries was filled with matrix. Chronic treatment of the rats with rGLP-1 significantly reduced the degree of matrix expansion and the glomerular injury (FIG. 3B). For comparison, we also examined the degree of glomerular injury in a group of normotensive Dahl S rats maintained on a low salt diet for 14 days. The glomerular injury score was not significantly different from that seen in Dahl S rats that were treated with rGLP-1 and fed the high salt diet (2.5±0.04 vs. 2.64±0.05, FIG. 3B). These results are consistent with previous reports that Dahl S rats exhibit a high degree of glomerular damage even when maintained on a low salt diet to minimize the development of hypertension [26]. Similarly, there was marked necrosis of renal tubules and formation of protein casts in the outer medulla of vehicle-treated rats (FIG. 3C). In contrast, the number of protein casts and the degree of tubular necrosis was greatly reduced in the outer medulla of Dahl S rats infused with rGLP-1 (FIG. 3C).

### Example 4

### Improvement in Endothelial Function

Thoracic aorta of vehicle- and GLP-1-treated rats were collected and placed in cold physiological saline solution (PSS) containing (in mmol/l): 119 NaCL, 4.7 KCl, 1.17 MgSO₄, 1.6 CaCl₂. 12 NaHCO₃, 1.18 NaH₂PO₄, 0.03 EDTA, 10 glucose and 10 HEPES (pH 7.4). The connective tissue and two rings (about 5 mm in length) were prepared from the aorta of each rat. The rings were mounted in an organ bath on tungsten wires connected to force transducers (Model FT03E, Grass Instruments, Rhode Island). The vessels were bathed in PSS, bubbled with 95% O₂ and 5% CO₂ and maintained at 37° C. Data were acquired using a computerized data acquisition system (WINDAQ software). The rings were preloaded with 2-3 g tension and were allowed to equilibrate for 60-90 min until a reproducible contraction was achieved following addition of a depolarizing concentration of 60 mmol/l KCl to the bath. Vessels were preconstriced with norepinephrine (NE, 10⁻⁷ mol/l). Then, cumulative dose-response curves to acetylcholine (Ach, from 10⁻⁹ to 10⁻⁴ mol/l) or a NO donor, DEA NON-Oate (from 10⁻⁹ to 10⁻⁴ mol/l), were constructed. Between each dose-response study, rings were bathed in fresh PSS and re-equilibrated for 60 min. Control experiments were performed on aortic rings from a group of normotensive Sprague-Dawley (SD) rats.

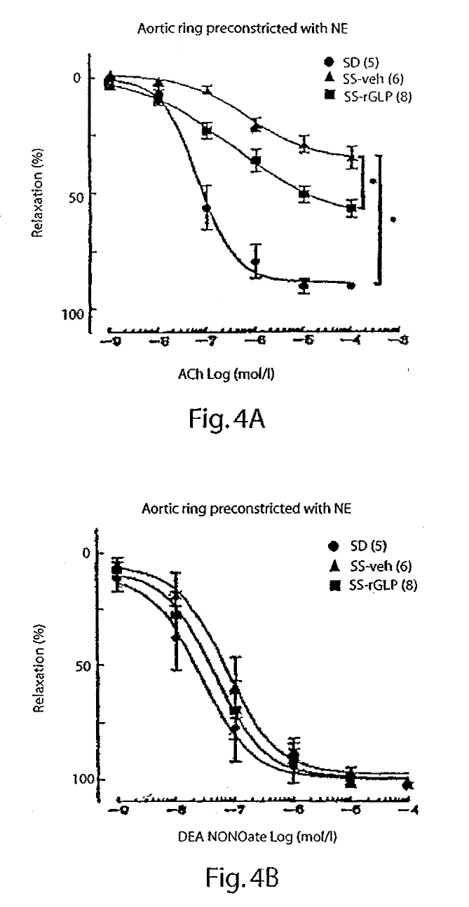

FIG. 4 indicates the effects of GLP-1 to help restore endothelial function. In normotensive SD rats, Ach reduced the tension of aortic ringes preconstricted with NE by 90%. The vasodilator response to ACh was markedly less in aortic rings prepared from vehicle-treated Dahl S rats fed a high salt diet. Chronic treatment of the Dahl S rats with GLP-1 partially restored the endothelial function. The vasodilator response to Ach in the GLP-1 treated rats was nearly twice that seen in the vehicle-treated raths (57±4 vs. 35±5% relaxation at 10⁻⁴M, FIG. 4a). The vasodilator responses to the NO donor were similar across aortic rings from all rats (FIG. 4b).

These examples demonstrate that an exemplary molecule of the invention, GLP-1, has antihypertensive and renoprotective effects.

From the above example it can be seen that the invention accomplishes its stated objectives. Changes in the methodology can be made without departing from the spirit and scope of the invention. The contents of patents referenced herein are all incorporated by reference.

What is claimed is:

1. A method for treating a subject having nephropathy comprising:
treating nephropathy by administering to an individual in need of such treatment a composition consisting of an effective amount of exendin-3 or exendin-4, an analog or derivative thereof, having at least 90% amino acid sequence homology to exendin-3 or exendin-4.

2. The method of claim 1 wherein said exendin-3 or exendin-4 agonist analog is 90% identical to SEQ ID NO: 13.

3. The method of claim 1 wherein the compound is administered parenterally.

4. The method of claim 3 wherein the compound is administered intravenously in a dose of 0.1 pmol/kg/min to 10 pmol/kg/min.

5. The method of claim 1 wherein the compound is administered subcutaneously in a dose of 0.1 pmol/kg/min to 75 pmol/kg/min.

6. A method for preventing or treating progression of End Stage Renal Disease in a subject having nephropathy comprising treating end stage renal disease by administering to an individual in need of such treatment a composition consisting of an effective amount of an exendin, an analog or derivative thereof having at least 90% amino acid sequence homology to an exendin.

7. The method of claim 10 wherein said exendin agonist analog is 90% identical to SEQ ID NO: 13.

8. The method of claim 6 wherein the compound is administered parenterally.

9. The method of claim 8 wherein the compound is administered intravenously in a dose of 0.1 pmol/kg/min up to 10 pmol/kg/min.

10. The method of claim 6 wherein the compound is administered subcutaneously in a dose of 0.1 pmol/kg/min to 75 pmol/kg/min.

11. A method for preventing or slowing progression of glomerulosclerosis in a subject comprising
preventing or slowing progression of glomerulosclerosis by administering to an individual in need of such treatment a composition consisting of an effective amount of an exendin, an analog or derivative thereof, having at least 90% amino acid sequence homology to an exendin.

12. The method of claim 11 wherein said exendin agonist analog is 90% identical to SEQ ID NO: 13.

13. The method of claim 11 wherein the compound is administered parenterally.

14. The method of claim 13 wherein the compound is administered intravenously in a dose of 0.1 pmol/kg/min to 10 pmol/kg/min.

15. The method of claim 13 wherein the compound is administered subcutaneously in a dose of 0.1 pmol/kg/min to 75 pmol/kg/min.


SEQUENCE LISTING (34 sequences)

SEQ ID NO: 1 - protein, 30 aa, Artificial Sequence
  GLP-1 (7-36)
HAEGTFTSDVSSYLEGQAAKEFIAWLVKGR

SEQ ID NO: 2 - protein, 31 aa, artificial sequence
  artificial sequence with specific variable residues
XAEGTFTSDVSSYLEGQAAXEFIAWLVKGRX

SEQ ID NO: 3 - protein, 21 aa, artificial sequence
  artificial sequence with specific variable residues
XSYLEGQAAKEFIAWLVXGRX

SEQ ID NO: 4 - protein, 21 aa, artificial sequence
  artificial sequence with specific variable residues
XSYLEGQAAKEFIAWLVXGRX

SEQ ID NO: 5 - protein, 21 aa, artificial sequence
  artificial sequence with specific variable residues
XSYLEGQAAKEFIAWLVXGRX

SEQ ID NO: 6 - protein, 21 aa, artificial sequence
  artificial sequence with specific variable residues
XSYLEGQAAKEFIAWLVXGRX

SEQ ID NO: 7 - protein, 21 aa, artificial sequence
  artificial sequence with specific variable residues
XSYLEGQAAKEFIAWLVXGRX

SEQ ID NO: 8 - protein, 21 aa, artificial sequence
  artificial sequence with specific variable residues
XSYLEGQAAKEFIAWLVXGRX

SEQ ID NO: 9 - protein, 21 aa, artificial sequence
  artificial sequence with specific variable residues
XSYLEGQAAKEFIAWLVXGRX

SEQ ID NO: 10 - protein, 21 aa, artificial sequence
  artificial sequence with specific variable residues
XSYLEGQAAKEFIAWLVXGRX

SEQ ID NO: 11 - protein, 31 aa, artificial sequence
  artificial sequence with specific variable residues
HAEGTFTSDVSSYLEGQAAKEFIAWLVKXXX

SEQ ID NO: 12 - protein, 39 aa, Heloderma horridum
  MOD_RES (39): AMIDATION, Position 39 is Ser-NH2
HSDGTFTSDLSKQMEEEAVRLFIEWLKNGGPSSGAPPPS

SEQ ID NO: 13 - protein, 31 aa, Artificial Sequence
  artificial sequence with specific variable residues
DLSKQMEEEAVRLFIEWLKNGGPSSGAPPPS

SEQ ID NO: 14 - protein, 39 aa, Heloderma suspectum
  MOD_RES (39): AMIDATION, Position 39 is Ser-NH2
HGEGTFTSDLSKQMEEEAVRLFIEWLKNGGPSSGAPPPS

SEQ ID NO: 15 - protein, 38 aa, Artificial Sequence
  artificial sequence with specific variable residues
HSDATFTAEYSKLLAKLALQKYLESILGSSTSPRPPSS

SEQ ID NO: 16 - protein, 37 aa, Artificial Sequence
  artificial sequence with specific variable residues
HSDATFTAEYSKLLAKLALQKYLESILGSSTSPRPPS

SEQ ID NO: 17 - protein, 35 aa, Artificial Sequence
  artificial sequence with specific variable residues
HSDAIFTEEYSKLLAKLALQKYLASILGSRTSPPP

SEQ ID NO: 18 - protein, 35 aa, Artificial Sequence
  artificial sequence with specific variable residues
HSDAIFTQQYSKLLAKLALQKYLASILGSRTSPPP

SEQ ID NO: 19 - protein, 30 aa, Artificial sequence
  Exendin-4 (1-30)
HGEGTFTSDLSKQMEEEAVRLFIEWLKNGG

SEQ ID NO: 20 - protein, 30 aa, Artificial sequence; identical to SEQ ID NO: 19
  Exendin-4 (1-30) Amide

SEQ ID NO: 21 - protein, 28 aa, Artificial Sequence
  Exendin-4 (1-28) Amide
HGEGTFTSDLSKQMEEEAVRLFIEWLKN

SEQ ID NO: 22 - protein, 39 aa, Artificial sequence
  14-Leu, 25-Phe form of exendin-4
HGEGTFTSDLSKQLEEEAVRLFIEFLKNGGPSSGAPPPS

SEQ ID NO: 23 - protein, 28 aa, Artificial sequence
  Truncated form of 14-leu, 25-Phe exendin-4
HGEGTFTSDLSKQLEEEAVRLFIEFLKN

SEQ ID NO: 24 - protein, 28 aa, Artificial Sequence
  14-Leu, 22-Ala, 25-Phe form of exendin-4(1-28)
HGEGTFTSDLSKQLEEEAVRLAIEFLKN

SEQ ID NO: 25 - protein, 29 aa, Artificial Sequence
  Formula III  artificial sequence with specific variable residues
XXXGXXXXXXXXXXXXXAXXXXXXXXXXX

SEQ ID NO: 26 - protein, 29 aa, Artificial Sequence
  Formula IV Artificial sequence with specific variable residues
XXXXXXXXXXXXXXXXXAXXXXXXXXXXX

SEQ ID NO: 27 - protein, 29 aa, artificial sequence
  Formula V Artificial sequence with specific variable residues
XXXGXXXXXXXXXXXXXAXXXXXXXXXXX

SEQ ID NO: 28 - protein, 29 aa, Artificial Sequence
  Formula VIArtificial sequence with specific variable residues
XXXXXXXXXXXXXXXXXAXXXXXXXXXXX

SEQ ID NO: 29 - protein, 28 aa, Artificial Sequence
  Formula VII Artificial sequence with specific variable residues
XXXGXXXXXXXXXXXXXAXXXXXXXXXX

SEQ ID NO: 30 - protein, 29 aa, Artificial sequence
  Formula VIII Artificial sequence with specific variable residues
XXXXXXXXXXXXXXXXXAXXXXXXXXXXX

SEQ ID NO: 31 - protein, 39 aa, Artificial sequence
  Formula IX artificial sequence with specific variable residues
XXXGTXXXXXSKQXEEEAVRLXXXXLKNGGXSSGAXXXX

SEQ ID NO: 32 - protein, 38 aa, Artificial sequence
  Formula X artificial sequence with specific variable residues
XXXGTXXXXXSKQXEEEAVRLXXXXLXGGXSSGAXXXX

SEQ ID NO: 33 - protein, 31 aa, artificial sequence
  Formula XI artificial sequence with specific variable residues
XXEGTFTSDVSSYLXGQAAKXFIAWLVKGRX

SEQ ID NO: 34 - protein, 31 aa, artificial sequence; identical to SEQ ID NO: 33
  Formula XII artificial sequence with specific varibale residues